(19) United States
(12) Patent Application Publication
(10) Pub. No.: US 2003/0162762 A1
(43) Pub. Date: Aug. 28, 2003

(54) Novel cephalosporin compounds and process for preparing the same

(76) Inventors: Chang-Seok Lee, Yuseong-ku (KR); Seong-Ho Oh, Yuseong-ku (KR); Eun-Jung Ryu, Yuseong-ku (KR); Hyung-Yeul Joo, Yuseong-ku (KR); Ha-Sik Youn, Yuseong-ku (KR); Yong-Jin Jang, Yuseong-ku (KR); Geun-Tae Kim, Yuseong-ku (KR)

Correspondence Address: BIRCH STEWART KOLASCH & BIRCH, PO BOX 747, FALLS CHURCH, VA, 22040-0747, US

(21) Appl. No.: 10/276,961

(22) PCT Filed: Jun. 14, 2001
(86) PCT No.: PCT/KR01/01027

(30) Foreign Application Priority Data
Jul. 7, 2000 (KR) 2000/38801

(51) Int. Cl. A61K 31/545; C07D 501/14

(52) U.S. Cl. 514/202; 540/225; 540/222

(57) ABSTRACT

The present invention relates to a novel cephalosporin compound, and pharmaceutically acceptable non-toxic salt, physiologically hydrolysable ester, hydrate, solvate or isomer thereof, to a pharmaceutical composition containing the compound and to a process for preparing the compound.

### TECHNICAL FIELD

The present invention relates to a novel cephalosporin compound useful as an antibiotic agent. More specifically, the present invention relates to a novel cephalosporin compound represented by the following formula (I), which is useful as an antibacterial agent, and particularly, exhibits a potent activity against strains such as methicillin-resistant Staphylococcus aureus (MRSA):

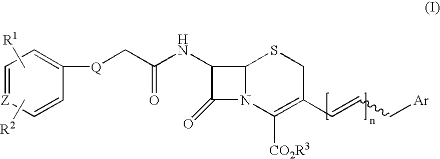

and pharmaceutically acceptable non-toxic salt, physiologically hydrolysable ester, hydrate, solvate or isomer thereof, in which

R¹ and R² independently of one another represent hydrogen, halogen, C₁₋₆ alkyl, C₁₋₆ alkylthio, aryl, arylthio, or C₅₋₆ heteroaryl containing one or two hetero atoms selected from the group consisting of nitrogen and oxygen;

R³ represents hydrogen or a carboxy-protecting group;

Q represents O, S, CH₂, NH or NR, wherein R represents hydrogen, C₁₋₆ alkyl or benzyl;

Z represents CH or N;

n denotes an integer of 0 or 1;

Ar represents a heteroaryl group represented by one of the following formulas:

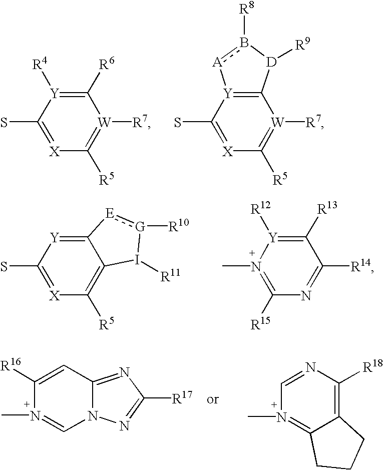

wherein X, Y, W, A, B, D, E, G and I independently of one another represent N or C (or CH), provided that the six-membered ring forms a pyrimidine structure;

R⁴ represents hydrogen or C₁₋₄ alkyl or amino substituted or unsubstituted with a substituent selected from the group consisting of C₁₋₆ alkyl and C₁₋₆ hydroxyalkyl;

R⁵ and R⁶ independently of one another represent hydrogen or hydroxy, or represent C₁₋₄ alkyl, C₅₋₆ alkylthio or amino substituted or unsubstituted with a substituent selected from the group consisting of C₁₋₆ alkyl, C₁₋₆ hydroxyalkyl and C₁₋₆ aminoalkyl;

R⁷, R⁸, R⁹, R¹⁰ and R¹¹ independently of one another represent hydrogen, or represent C₁₋₆ alkyl, or represent amino substituted or unsubstituted with a substituent selected from the group consisting of C₁₋₆ alkyl, C₁₋₆ hydroxyalkyl and C₁₋₆ aminoalkyl;

R¹², R¹³, R¹⁴, R¹⁵, R¹⁶, R¹⁷ and R¹⁸ independently of one another represent hydrogen, C₁₋₆ alkyl or C₁₋₆ hydroxyalkyl, or represent amino substituted or unsubstituted with a substituent selected from the group consisting of C₁₋₆ alkyl, di-C₁₋₆ alkyl, C₁₋₆ hydroxyalkyl and C₁₋₆ aminoalkyl;

[symbol] denotes a single bond or a double bond; and

the propenyl group when n is 1 at C-3 position may be present in the form of cis or trans.

The present invention also relates to a process for preparing the compound of formula (I), as defined above, and to an antibacterial composition containing the compound of formula (I) as an active ingredient.

### BACKGROUND ART

Cephalosporin-based antibiotics have been widely used for treatment of infectious diseases caused by pathogenic bacteria in human and animals. They are particularly useful for treatment of diseases caused by bacteria resistant to other antibiotics such as penicillin compounds and for treatment of penicillin-hypersensitive patients. In most cases for treating such infectious diseases, it is preferred to use antibiotics showing an antimicrobial activity against both of gram-positive and gram-negative microorganisms. It has been very well known that such antimicrobial activity of cephalosporin antibiotics is largely influenced by the kind of substituents present at 3- or 7-position of cephem ring. Therefore, according to the attempt to develop an antibiotic agent showing a potent antimicrobial activity against broad strains of gram-positive and gram-negative bacteria numerous cephalosporin antibiotics having various substituents introduced into 3- or 7-position have been developed up to the present.

For instance, British Patent No. 1,399,086 illustrates broadly and generically cephalosporin derivatives represented by the following formula (II):

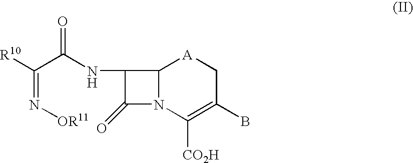

in which

R¹⁰ represents hydrogen or an organic group;

R¹¹ is an etherified monovalent organic group, which is linked to oxygen via carbon atom;

A represents —S— or >S→O; and

B represents an organic group.

Since development of those compounds, many attempts to develop antibiotic agents having broad antibacterial spectrum have been made and, as a result, numerous cephalosporin antibiotics have been developed. According to their development, many studies to introduce acylamido group into 7-position and a certain specific group into C-3 position of the cephem nucleus of formula (II) have also been made in various points of view.

Recently, resistance strains of gram-positive microorganisms, particularly methicillin-resistant Staphylococcus aureus (MRSA) have been recognized as the cause of serious hospital infection and therefore, many attempts have been made to introduce arylthio group into C-3 position to develop cephalosporin compounds showing a potent activity against MRSA.

Thus, Japanese Laid-open Publication No. 98-36375 discloses broadly and generically cephalosporin derivatives represented by the following formula (III) wherein arylthio group is introduced into C-3 position to increase the activity against broad pathogenic strains:

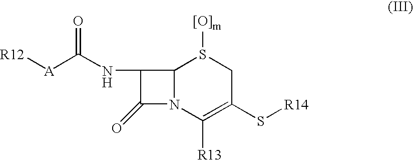

in which

R¹² represents substituted alkylthio, aryl, arylthio, aryloxy or heterocyclyl group;

A represents protected amino, hydroxy or methylene group;

R¹³ represents protected carboxy or carboxylate;

R¹⁴ represents halo, cyano, amidino, guanidino, azido, nitro, substituted alkyl, alkenyl, dichloroalkyl, aryl, alkoxy, aryloxy, alkylthio, arylthio, alkylamino, acyl, carbamoyl, carbamoyloxy, alkoxyimino, ureido, alkylsulfinyl, alkylsulfonyl or sulfamoyl, or 2-substituted pyrimidinyl, quinazolinyl, purinyl, pyrazolo[3,4-d]pyrimidinyl, pyrazolo[4,3-d]pyrimidinyl, [1,2,3]triazolo[4,5-d] pyrimidinyl or phtheridinyl; and

m denotes 0 or 1.

In the above patent various heteroaromatic rings are introduced into thioaryl moiety at C-3 position, but are different from the methylene or propenyl chain at C-3 position of the compound according to the present invention.

In other words, the present invention characterized in that substituted or unsubstituted pyrimidinyl group is introduced into C-3 position via a chain such as methylene or propenyl, but the above Japanese patent mentions nothing thereon.

The attempt has been made to develop cephalosporin compounds, which can show a potent activity against serious hospital infection caused by methicillin-resistant Staphylococcus aureus (MRSA), by introducing acyl group into position 7 and pyridine group into C-3 position. Typical example thereof is the compounds of formula (IV) disclosed in European Patent No. EP 96-72742 A1:

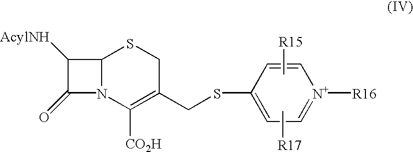

in which

Acyl substituent is Ar—S—CH₂—CO—, wherein Ar represents hydrophobic substituted phenyl, pyridyl or benzthiazolyl group;

R¹⁵ and R¹⁶ independently of one another represent hydrogen, alkyl or aminoalkyl-carbonylamino; and

R¹⁷ represents substituted aliphatic, aromatic or arylaliphatic group or a group containing sugar moiety.

In the above European patent, various heteroaromatic rings are introduced into thioaryl moiety present at C-3 position but are different from the substituent present at C-3 position of the compound according to the present invention.

Another attempt has been made to develop cephalosporin compounds, which can show a potent activity against serious hospital infection caused by methicillin-resistant Staphylococcus aureus (MRSA), by introducing acyl group into position 7 and quarternary ammonium group into C-3 position via propenyl chain. Typical example thereof is the compounds of formula (IVa) disclosed in WO99/67255:

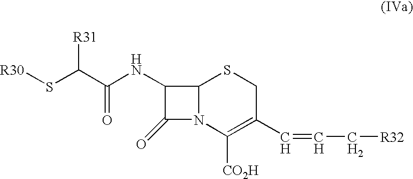

in which

R³⁰ represents an organic group having a molecular weight of 400 or less;

R³¹ represents hydrogen, lower alkyl or phenyl group; and

R³² represents an organic group of which secondary, tertiary or quarternary nitrogen atom is directly connected with propenyl group, and which has a molecular weight of 400 or less.

In the above patent, an organic group is introduced via various nitrogen atoms into propenyl moiety present at C-3 position but are quite different from the substituent present at C-3 position of the compound according to the present invention.

That is, the present invention characterized in that substituted or unsubstituted pyrimidinyl group is introduced into C-3 position via a chain such as methylene or propenyl, but the above patent mentions nothing thereon.

### DISCLOSURE OF INVENTION

Thus, the present inventors have conducted extensive and intensive researches to develop cephalosporin compounds showing broad antibacterial activity against gram-positive microorganisms including MRSA. As a result, we have identified that a certain cephalosporin compound having optionally substituted pyrimidinyl group at C-3 position meets the above requirement, and then completed the present invention.

Therefore, the purpose of the present invention is to provide a compound of formula (I), as defined above, and pharmaceutically acceptable non-toxic salt, physiologically hydrolysable ester, hydrate, solvate or isomer thereof.

Further, the purpose of the present invention is to provide a process for preparing the compound of formula (I) and an antibacterial composition containing the compound of formula (I) as an active ingredient.

### BEST MODE FOR CARRYING OUT THE INVENTION

The purpose of the present invention is to provide a novel cephalosporin compound represented by the following formula (I):

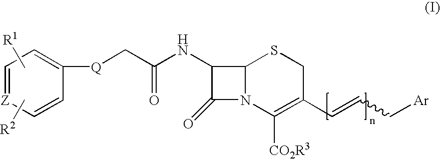

and pharmaceutically acceptable non-toxic salt, physiologically hydrolysable ester, hydrate, solvate or isomer thereof, in which

R¹ and R² independently of one another represent hydrogen, halogen, C₁₋₆ alkyl, C₁₋₆ alkylthio, aryl, arylthio, or C₅₋₆ heteroaryl containing one or two hetero atoms selected from the group consisting of nitrogen and oxygen;

R³ represents hydrogen or a carboxy-protecting group;

Q represents O, S, CH₂, NH or NR, wherein R represents hydrogen, C₁₋₆ alkyl or benzyl;

Z represents CH or N;

n denotes an integer of 0 or 1;

Ar represents a heteroaryl group represented by one of the following formulas:

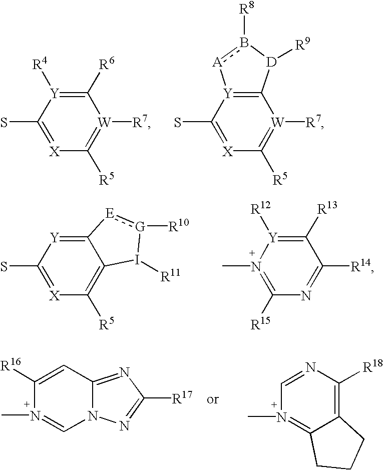

wherein X, Y, W, A, B, D, E, G and I independently of one another represent N or C (or CH), provided that the six-membered ring forms a pyrimidine structure;

R⁴ represents hydrogen or C₁₋₄ alkyl, or amino substituted or unsubstituted with a substituent selected from the group consisting of C₁₋₆ alkyl and C₁₋₆ hydroxyalkyl;

R⁵ and R⁶ independently of one another represent hydrogen or hydroxy, or represent C₁₋₄ alkyl, C₁₋₆ alkylthio or amino substituted or unsubstituted with a substituent selected from the group consisting of C₁₋₆ alkyl, C₁₋₆ hydroxyalkyl and C₁₋₆ aminoalkyl;

R⁷, R⁸, R⁹, R¹⁰ and R¹¹ independently of one another represent hydrogen, or represent C₁₋₆ alkyl, or represent amino substituted or unsubstituted with a substituent selected from the group consisting of C₁₋₆ alkyl, C₁₋₆ hydroxyalkyl and C₁₋₆ aminoalkyl;

R¹², R¹³, R¹⁴, R¹⁵, R¹⁶, R¹⁷ and R¹⁸ independently of one another represent hydrogen, C₁₋₆ alkyl or C₁₋₆ hydroxyalkyl, or represent amino substituted or unsubstituted with a substituent selected from the group consisting of C₁₋₆ alkyl, di-C₁₋₆ alkyl, C₁₋₆ hydroxyalkyl and C₁₋₆ aminoalkyl;

[symbol] denotes a single bond or a double bond; and

the propenyl group when n is 1 at C-3 position may be present in the form of cis or trans.

The compound of formula (I) according to the present invention can be administered in the form of an injectable formulation or an oral formulation depending on the purpose of its use.

Pharmaceutically acceptable non-toxic salts of the compound of formula (I) include salts with inorganic acids such as hydrochloric acid, hydrobromic acid, phosphoric acid, sulfuric acid, etc., salts with organic carboxylic acids such as acetic acid, trifluoroacetic acid, citric acid, formic acid, maleic acid, oxalic acid, succinic acid, benzoic acid, tartaric acid, fumaric acid, mandelic acid, ascorbic acid, malic acid, etc., or with methanesulfonic acid or para-toluenesulfonic acid, and salts with other acids which have been well-known and widely used in the technical field of penicillins and cephalosporins. These acid addition salts can be prepared according to any of the conventional methods. Further, the compound of formula (I) can also form a non-toxic salt with a base. The base that can be used for this purpose includes inorganic bases such as alkaline metal hydroxides (e.g. sodium hydroxide, potassium hydroxide, etc.), alkaline metal bicarbonates (e.g. sodium bicarbonate, potassium bicarbonate, etc.), alkaline metal carbonates (e.g. sodium carbonate, potassium carbonate, calcium carbonate, etc.), etc., and organic bases such as amino acids.

Examples of physiologically hydrolysable esters of the compound of formula (I) include indanyl, phthalidyl, methoxymethyl, pivaloyloxymethyl, glycyloxymethyl, phenylglycyloxymethyl, 5-methyl-2-oxo-1,3-dioxolen-4-yl methyl esters or other physiologically hydrolysable esters which have been well-known and widely used in the field of penicillins and cephalosporins. These esters can be prepared according to any of the known conventional methods.

Typical examples of the compound of formula (I) according to the present invention include the following:

I-1:

(6R,7R)-3-{(E)-3-[(2-amino-6-hydroxy-4-pyrimidinyl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid;

I-2:

4-amino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}pyrimidin-1-ium;

I-3:

(6R,7R)-3-{(E)-3-[(2,6-diamino-4-pyrimidinyl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid;

I-4:

1,4-diamino-2-({(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}sulfanyl) pyrimidin-1-ium;

I-5:

(6R,7R)-3-{(E)-3-[(2,6-diamino-4-pyrimidinyl)sulfanyl]-1-propenyl-7-{[2-(2,5-dichloroanilino)acetyl]amino}-8-oxo-5-thia-1-azabicyclo[4.2.0]oct-2-ene-2-carboxylic acid;

I-6:

1,4-diamino-2-({[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]methyl}sulfanyl)pyrimidin-1-ium;

I-7:

(6R,7R)-7-({2-[(2,6-dichloro-4-pyridinyl)sulfanyl]acetyl}amino)-8-oxo-3-[(E)-3-(1H-pyrazolo[3,4-d]pyrimidin-4-ylsulfanyl]-1-propenyl]-5-thia-1-azabicyclo[4.2.0]oct-2-ene-2-carboxylic acid;

I-8:

(6R,7R)-3-{(E)-3-[(4,6-diamino-2-pyrimidinyl)sulfanyl]-1-propenyl}-7-({2-[(2,6-dichloro-4-pyridinyl)sulfanyl]acetyl}amino)-8-oxo-5-thia-1-azabicyclo[4.2.0]oct-2-ene-2-carboxylic acid;

I-9:

(6R,7R)-3-{(E)-3-[(4-amino-1H-pyrazolo[3,4-d]pyrimidin-6-yl)sulfanyl]-1-propen yl}-7-({2-[(2,6-dichloro-4-pyridinyl)sulfanyl]acetyl}amino)-8-oxo-5-thia-1-azabicyclo[4.2.0]oct-2-ene-2-carboxylic acid;

I-10:

(6R,7R)-3-{(E)-3-[(2-amino-6-hydroxy-4-pyrimidinyl)sulfanyl]-1-propenyl}-7-({2-[(2,6-dichloro-4-pyridinyl)sulfanyl]acetyl}amino)-8-oxo-5-thia-1-azabicyclo [4.2.0]oct-2-ene-2-carboxylic acid;

I-11:

(6R,7R)-3-{(E)-3-[(2,6-diamino-4-pyrimidinyl)sulfanyl]-1-propenyl}-7-( {2-[(2,6-dichloro-4-pyridinyl)sulfanyl]acetyl}amino)-8-oxo-5-thia-1-azabicyclo[4.2.0]oct-2-ene-2-carboxylic acid;

I-12:

(6R,7R)-7-({2-[(2,6-dichloro-4-pyridinyl)sulfanyl]acetyl}amino)-3-((E)-3-{[2-(ethylsulfanyl)-6-methyl-4-pyrimidinyl]sulfanyl}-1-propenyl)-8-oxo-5-thia-1-azabicyclo[4.2.0]oct-2-ene-2-carboxylic acid;

I-13:

7-amino-5-({(E)-3-[(6R,7R)-2-carboxy-7-({2- [(2,6-dichloro-4-pyridinyl)sulfanyl]acetyl}amino)-8-oxo-5-thia-1-azabicyclo[4.2.0]oct-2-en-3-yl]-2-propenyl}sulfanyl) -1H-[1,2,4]triazolo[1,5-c]pyrimidin-4-ium;

I-14:

2,7-diamino-5-({(E)-3-[(6R,7R)-2-carboxy-7-({2-[(2,6-dichloro-4-pyridinyl) sulfanyl]acetyl}amino)-8-oxo-5-thia-1-azabicyclo[4.2.0]oct-2-en-3-yl]-2-propenyl}sulfanyl)-1-methyl-1H-[1,2,4]triazolo[1,5-c]pyrimidin-4-ium;

I-15:

(6R,7R)-7-({2-[(2,6-dichloro-4-pyridinyl)sulfanyl]acetyl}amino)-3-[(E)-3-({4-hydroxy-6-[(2-hydroxyethyl)amino]-2-pyrimidinyl]sulfanyl }-1-propenyl]-8-oxo-5-thia-1-azabicyclo[4.2.0]oct-2-ene-2-carboxylic acid;

I-16:

4,6-diamino-2-({(E)-3-[(6R,7R)-2-carboxy-7-({2-[(2,6-dichloro-4-pyridinyl) sulfanyl]acetyl}amino)-8-oxo-5-thia-1-azabicyclo[4.2.0]oct-2-en-3-yl]-2-propenyl}sulfanyl)-1-ethylpyrimidin-1-ium;

I-17:

1,2-diamino-4-({(E)-3-[(6R,7R)-2-carboxy-7-({2-[(2,6-dichloro-4-pyridinyl) sulfanyl]acetyl}amino)-8-oxo-5-thia-1-azabicyclo[4.2.0]oct-2-en-3-yl]-2-propenyl}sulfanyl)-6,7-dihydro-5H-cyclopenta[d]pyrimidin-1-ium;

I-18:

4,6-diamino-1-({(E)-3-[(6R,7R)-2-carboxy-7-({2-[(2,6-dichloro-4-pyridinyl) sulfanyl]acetyl}amino)-8-oxo-5-thia-1-azabicyclo[4.2.0]oct-2-en-3-yl]-2-propenyl}pyrimidin-1-ium;

I-19:

(6R,7R)-7-amino-5-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}sulfanyl)-3H-[1, 2,4]triazolo[1,5-c]pyrimidin-4-ium;

I-20:

(6R,7R)-3-{(E)-3-[(4-amino-6,7-dihydro-5H-cyclopenta[d]pyrimidin-2-yl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid,

I-21:

(6R,7R)-1,2-diamino-4-({(E)-3-[2-carboxy-7-({2-[(2,5-dichlorophenyl)sulfanyl]acetyl}amino-8-oxo-5-thia-1-azabicyclo[4.2.0]oct-2-en-3 -yl-2-propenyl}sulfanyl)-6,7-dihydro-5H-cyclopenta[d]pyrimidin-1-ium;

I-22:

(6R,7R)-2,6-diamino-4-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}sulfanyl)-1-methylpyrimidin-1-ium;

I-23:

(6R,7R)-4,6-diamino-2-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}sulfanyl)-1-methyl-5-[(methylamino)methyl]pyrimidin-1-ium;

I-24:

(6R,7R)-3-{(E)-3-[(4,6-diamino-2-pyrimidinyl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid;

I-25:

(6R,7R)-3-{(E)-3-[(5,6-diamino-4-pyrimidinyl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid;

I-26:

(6R,7R)-3-{(E)-3-[(4,6-diamino-5-methyl-2-pyrimidinyl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid;

I-27:

(6R,7R)-7-({2-[(2,5-dichlorophenyl)sulfanyl]acetyl}amino)-8-oxo-3-[(E)-3-(1H-pyrazolo[3,4-d]pyrimidin-4-ylsulfanyl)-1-propenyl-5-thia-1-azabicyclo[4.2.0]oct-2-ene-2-carboxylic acid;

I-28:

(6R,7R)-7-({2-[(2,5-dichlorophenyl)sulfanyl]acetyl}amino)-3-((E)-3-{[6-methyl-2-(methylsulfanyl)-4-pyrimidinyl]sulfanyl}-1propenyl)-8-oxo-5-thia-1-azabicyclo [4.2.0]oct-2-ene-2-carboxylic acid;

I-29:

(6R,7R)-4,6-diamino-2-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}sulfanyl)-1-ethylpyrimidin-1-ium;

I-30:

(6R,7R)-3-((E)-3-{[4-amino-6-(methylamino)-2-pyrimidinyl]sulfanyl}-1propenyl)-7-({2-[(2,5-dichlorophenyl)sulfanyl]acetyl}amino)-8-oxo-5-thia-1-azabicyclo [4.2.0]oct-2-ene-2-carboxylic acid;

I-31:

(6R,7R)-4,6-diamino-1-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-5-methylpyrimidin-1-ium;

I-32:

(6R,7R)-2,7-diamino-6-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-[1,2,4]triazolo[1,5-c]pyrimidin-6-ium;

I-33:

(6R,7R)-4-amino-1-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-2-methyl pyrimidin-1-ium;

I-34:

(6R,7R)-4-amino-1-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-6,7-dihydro-5 H-cyclopenta[d]pyrimidin-1-ium;

I-35:

(6R,7R)-4,5,6-triamino-1-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}pyrimidin-1-ium;

I-36:

(6R,7R)-4,6-diamino-1-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}pyrimidin-1-ium;

I-37:

4-amino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-6-(dimethylamino)-2-methylpyrimidin-1-ium;

I-38:

4-amino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-2-methyl-6-(methylamino)pyrimidin-1-ium;

I-39:

4-amino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-2-methyl pyrimidin-1-ium;

I-40:

4,6-diamino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-5-methylpyrimidin-1-ium; and

I-41:

4-amino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-2-methyl-6-(methylamino)pyrimidin-1-ium.

According to the present invention, the compound of formula (I):

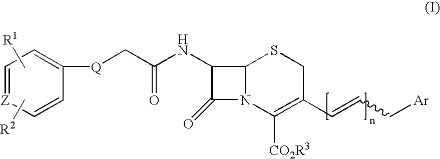

wherein R¹, R², R³, Z, Q, n and Ar are as defined above, and pharmaceutically acceptable non-toxic salt, physiologically hydrolysable ester, hydrate, solvate or isomer thereof can be prepared by a process which comprises reacting a compound of formula (V):

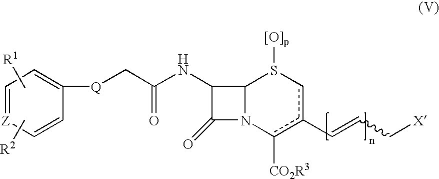

wherein R¹, R², R³, Z, Q and n are as defined in the formula (I), X′ represents halogen atom, and p is 0 or 1, with a compound of formula (VI):

H—Ar (VI)

wherein Ar is as defined in the formula (I), if necessary, after adding alkaline metal iodide, or if necessary, removing the acid-protecting group before or after the reaction, or reducing S→oxide of a compound of formula (VII):

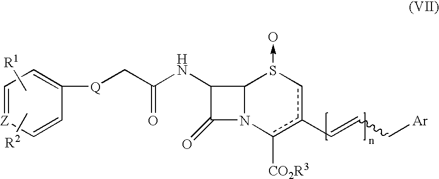

wherein R¹, R², R³, Z, Q, n and Ar are as defined in the formula (I).

The propenyl group as a part of C-3 substituent may be present as trans- or cis-isomeric form depending on the geometric arrangement around the double bond as follows:

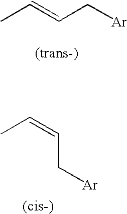

in which Ar is as defined above.

The present invention also includes the respective geometric isomers and mixtures thereof in its scope.

The process for preparing the compound of formula (I) by reacting the compound of formula (V) with the compound of formula (VI) according to the present invention may be carried out using an organic solvent. Suitable solvent for this purpose includes lower alkyl nitriles such as acetonitrile, propionitrile, etc., halogeno lower alkanes such as chloromethane, dichloromethane, chloroform, etc., ethers such as tetrahydrofuran, dioxane, ethyl ether, etc., amides such as dimethylformamide, etc., esters such as ethyl acetate, etc., ketones such as acetone, etc., hydrocarbons such as benzene, etc., alcohols such as methanol, ethanol, etc., sulfoxides such as dimethylsulfoxide, etc., or the mixtures thereof.

In the process for preparing the compound of formula (I) by reacting the compound of formula (V) with the compound of formula (VI) according to the present invention, the reaction temperature can be varied within a broad range and is generally in the range of −10° C. to 80° C., preferably in the range of 20° C. to 40° C.

In the case of carrying out the process according to the present invention, the compound of formula (VI) is used in an amount of 0.5 to 2 equivalents, preferably 1.0 to 1.1 equivalents with respect to the compound of formula (V).

In the above process, carboxy-protecting group R³ is desirably the group that can be readily removed under mild condition. Typical examples of carboxy-protecting group R³ include (lower)alkyl ester (e.g. methyl ester, t-butyl ester, etc.), (lower)alkenyl ester (e.g. vinyl ester, allyl ester, etc.), (lower)alkylthio(lower)alkyl ester (e.g. methylthiomethyl ester, etc.), halo(lower)alkyl ester (e.g. 2,2,2-trichloroethyl ester, etc.), substituted or unsubstituted aralkyl ester (e.g. benzyl ester, p-nitrobenzyl ester, p-methoxybenzyl ester, etc.) or silyl ester. These carboxy-protecting groups can be readily removed under mild reaction conditions such as hydrolysis, reduction, etc. to generate a free carboxy group, and appropriately selected depending on the chemical properties of the compound of formula (I).

The leaving group X′ represents halogen atom such as chloro, fluoro, iodo, etc.

The dotted line in the formulae in the present specification represents, for example, each of the following formulae (VIIIa) and (VIIIb), or their mixture:

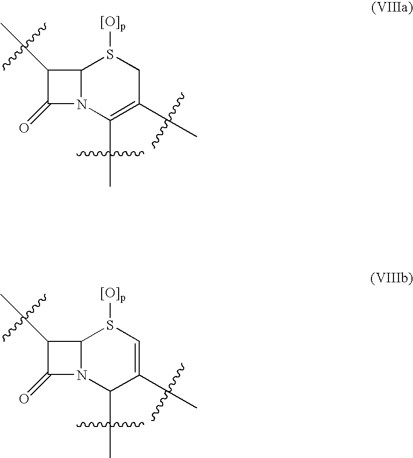

in which p is as defined above.

The compound of formula (V) can be prepared by activating a compound of formula (IX):

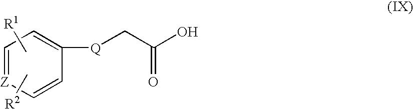

in which R¹, R², Z and Q are as defined above, or its salt with an acylating agent and then reacting the resulting activated compound with a compound of formula (X):

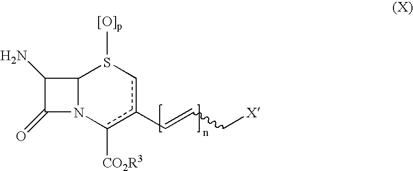

in which R³, n, p and X′ are as defined above.

In preparing the compound of formula (V), an acylated derivative as the activated form of the compound of formula (IX) includes acid chlorides, acid anhydrides, mixed acid anhydrides (preferably, acid anhydrides formed with methylchloroformate, mesitylene sulfonyl chloride, p-toluenesulfonyl chloride or chlorophosphate) or activated esters (preferably, esters formed from the reaction with N-hydroxybenzotriazole in the presence of a condensing agent such as dicyclohexylcarbodiimide), etc. In addition, the acylation reaction can also be practiced by using a free acid compound of formula (IX) in the presence of a condensing agent such as dicylcohexylcarbodiimide or carbonyldiimidazole. Further, the acylation reaction is well practiced generally in the presence of an organic base, preferably a tertiary amine such as triethylamine, dimethylaniline, pyridine, etc., or an inorganic base such as sodium bicarbonate, sodium carbonate, etc. The solvent which can be used in this reaction includes halogenated hydrocarbon such as methylene chloride, chloroform, etc., tetrahydrofuran, acetonitrile, dimethylformamide or dimethyl acetamide. The mixed solvent comprising two or more solvents selected from the above can be also used. The reaction can also be carried out in an aqueous solution.

The reaction temperature in the acylation reaction is in the range of −50° C. to 50° C., preferably in the range of −30° C. to 20° C. The acylating agent for the compound of formula (IX) can be used in an equimolar amount or a slightly excessive amount, i.e. in an amount of 1.05 to 1.5 equivalent weights, with respect to an equivalent weight of the compound of formula (X).

A compound of formula (Va) (wherein n is 1):

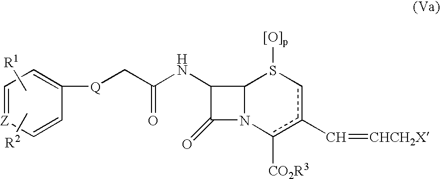

in which R¹, R², R³, Z, Q, p and X′ are as defined above, can be prepared according to a conventional method. That is, the compound of formula (Va) can be prepared by reacting a compound of formula (Vb) (wherein n is 0):

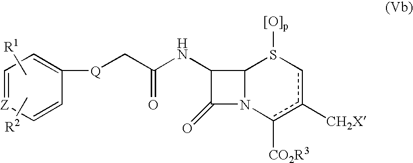

in which R¹, R², R, Z, Q, p and X′ are as defined above, according to a conventional method, e.g., Wittig reaction, to give an intermediate compound of formula (XI):

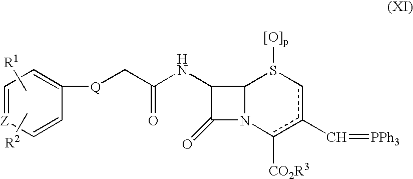

in which R¹, R², R³, Z, Q and p are as defined above, then by reacting the resulting compound (XI) with a halogenated acetaldehyde.

The compound of formula (V) above may also be prepared by acylating the compound of formula (IX) or its salt for activation, then by directly reacting the resulting acylated compound with the compound of formula (X).

Conversions of the halogen atom represented by X′ in formula (V) to another halogen atom may be carried out through a conventional method. For example, a compound of formula (V) wherein X′ is iodine atom is obtained by reacting a compound of formula (V) wherein X′ is chlorine atom with alkaline metal iodide.

In preparing the compound of formula (I) as defined above, the acid-protecting group present in the compound of formula (V) can be removed by any of the conventional methods widely known in the field of cephalosporins. That is, the protecting groups can be removed by hydrolysis or reduction. Acid hydrolysis is useful for removing tri(di)phenylmethyl group or alkoxycarbonyl group and is carried out using an organic acid such as formic acid, trifluoroacetic acid, p-toluenesulfonic acid, etc., or an inorganic acid such as hydrochloric acid, etc.

The resulting product from the above processes can be treated with various methods such as recrystallization, electrophoresis, silica gel column chromatography or ion exchange chromatography to separate and purify the desired compound of formula (I).

Another purpose of the present invention is to provide a pharmaceutical composition containing the compound of formula (I) or its pharmaceutically acceptable salt as an active ingredient, together with a pharmaceutically acceptable carrier.

The compound according to the present invention can be administered in the form of an injectable formulation or an oral formulation depending on the purpose of its use.

The compound of formula (I) of the present invention can be formulated using known pharmaceutically acceptable carriers and excipients according to the known method to prepare a unit dosage form or to be introduced into a multi-dosage container. The formulations can be in the form of a solution, suspension or emulsion in an oil or aqueous medium and can contain conventional dispersant, suspending agent or stabilizing agent. In addition, the formulation can also be in the form of a ready-to-use dry powder which can be used by dissolving with a sterile, pyrogen-free water before its use. The compound of formula (I) can also be formulated in the form of a suppository by using conventional suppository bases such as cocoa butter or other glycerides. Solid dosage form for oral administration includes capsules, tablets, pills, powders and granules, with capsules and tablets being particularly useful. For the tablets and pills, it is preferred to provide an enteric coating. Solid dosage form can be prepared by mixing the active compound of formula (I) according to the present invention with one or more inert diluents such as sucrose, lactose, starch, etc., and carriers including lubricants such as magnesium stearate, disintegrating agents, binders, etc.

If necessary, the compound of the present invention can be administered in combination with other antibacterial agent such as penicillins or other cephalosporins.

In formulating the compound of formula (I) according to the present invention into the unit dosage form, it is preferred that the unit dosage form contains the active ingredient of formula (I) in an amount of about 50 to 1,500 mg. The dosage of the compound of formula (I) is suitably selected under the physician's prescription depending on various factors including weight and age of patient, particular conditions and severity of diseases to be treated, etc. However, the daily dosage for treatment of adult man generally corresponds to about 500 to 5,000 mg of the compound of formula (I) depending on the frequency and intensity of administration. For intramuscular or intravenous injection to adult man, a total daily dosage in the range of about 150 to 3,000 mg is generally sufficient. However, in case of infections caused by some pathogenic strains, it may be preferred to more increase the daily doage.

The compound of formula (I) and its non-toxic salt (preferably salts with alkali metals, alkaline earth metals, inorganic acids, organic acids and amino acids) according to the present invention exhibit a potent antimicrobial activity and a broad antibacterial spectrum against broad pathogenic microorganisms including various gram-positive strains and therefore, are very useful for prevention and treatment of diseases caused by bacterial infection in animals including human being.

The present invention will be more specifically illustrated by the following preparations and examples. However, it should be understood that these preparations and examples are provided only to help the clear understanding of the present invention but do not intend to limit the present invention in any manner.

### EXAMPLES

### Preparation 1

### Synthesis of 4-methoxybenzyl (6R,7R)-3-[3-chloro-1-propenyl]-7-({2-1(2,5-dichlorophenyl)sulfanyl]acetyl}amino)-8-oxo-5-thia-1-azabicyclo[4.2.0]oct-2-ene-2-carboxylate

4-Methoxybenzyl (6R,7R)-7-amino-3-[3-chloro-1-propenyl]-8-oxo-5-thia-1-azabicyclo[4.2.0]oct-2-ene-2-carboxylate hydrochloride(2.73 g, 6.33 mmol) and 2,5-dichlorophenylthioacetic acid(1.50 g, 6.33 mmol) were dissolved in dichloromethane(25 ml). Temperature in the reaction vessel was lowered to −30° C., and each of pyridine(1.30 ml, 15.83 mmol) and phosphoryloxy chloride(0.71 ml, 7.60 mmol) was slowly added dropwise thereto. The temperature in the reaction vessel was gradually raised to 0° C. during which the reaction mixture was stirred for 3 hours. The reaction mixture was diluted with excess ethyl acetate, washed with saturated ammonium chloride solution, 5% aqueous sodium bicarbonate solution and aqueous sodium chloride solution once per each solution, dried over anhydrous magnesium sulfate, and filtered. The filtrate was distilled under reduced pressure and the residue was purified by column chromatography to give 1.8 g (Yield 46.3%) of the title compound.

¹HNMR(CDCl₃) δ 7.38˜7.25(4H, m), 7.15(1H, d), 6.88˜6.86(1H, q, J=1.85 Hz), 6.24˜6.22(1H, d, J=11 Hz), 5.75˜5.73(2H, dd, m), 5.15(2H, s), 4.98˜4.97(1H, d, J=5.05 Hz), 4.10(1H, m), 3.93˜3.90(1H, m), 3.79(3H, s), 3.75˜3,71(2H, q), 3.43(1H, Abq, J=18.3 Hz), 3.27˜3,23(1H, Abq, J=18.3Hz)

Mass(m/e) 612

### Example 1

### Synthesis of (6R,7R)-3-{(E)-3-[(2-amino-6-hydroxy-4-pyrimidinyl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid

4-Methoxybenzyl (6R,7R)-3-[3-chloro-1-propenyl]-7-({2-[(2,5-dichlorophenyl)sulfanyl]acetyl}amino)-8-oxo-5-thia-1-azabicyclo[4.2.0]oct-2-ene-2-carboxylate(0.2 g, 0.3263 mmol) was dissolved in acetone(3 ml) and sodium iodide(0.15 g, 0.9789 mmol) was added thereto. The reaction mixture was stirred for 1 hour at room temperature and the solvent was removed by distillation under reduced pressure. The resulting residue was dissolved in dimethylformamide(3 ml), 2-amino-4-hydroxy-6-mercaptopyrimidine ½ sulfate(0.044 g, 0.3099 mmol) was added thereto, and the mixture was stirred for 3 hours at room temperature. The reaction mixture was diluted with excess ethyl acetate, washed three times with aqueous sodium chloride solution, dried over anhydrous magnesium sulfate, and filtered. The filtrate was distilled under reduced pressure, and then the residue was purified by diethylether and dried under nitrogen atmosphere. Thus obtained solid(0.15 g) was deprotected by trifluoroacetic acid and anisole and then purified by high pressure preparative liquid chromatography to give 0.1 (Yield of two steps 27.7%) of the title compound.

¹HNMR(CD₃OD) δ 8.64(1H, s), 8.15˜8.13(1H, d, J=7.8 Hz), 7.46(1H, s), 6.69˜6.66(1H, d, J=15.6 Hz), 5.94˜5.91(1H, m), 5.69(1H, s), 5.50˜5.49(1H, d, J=4.6 Hz), 5.02˜5.01(1H, d, H=4.55 Hz), 3.72˜3.71(2H, q), 3.59˜3.52(2H, m)

Mass(m/e) 599

### Example 2

### Synthesis of 4-amino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}pyrimidin-1-ium

The title compound was prepared according to the same procedure as Example 1 (Yield of two steps 22.5%).

¹HNMR(CD₃OD) δ 8.64(1H, s), 8.15˜8.13(1H, d, J=7.8 Hz), 7.46(1H, s), 7.36˜7.35(1H, d, J=8.7 Hz), 7.18˜7.16(1H, dd, J=2.3 Hz), 7.02˜6.99(1H, d, J=15.5 Hz), 7.76˜7.74(1H, d, J=7.4 Hz), 5.90˜5.87(1H, m), 5.64˜5.63(1H, d, J=5.05 Hz), 5.02˜5.01(1H, d, Hz=5.0 Hz), 3.81˜3.73(2H, m), 3.59˜3.52(2H, m)

Mass(m/e) 552

### Example 3

### Synthesis of (6R,7R)-3-{(E)-3-[(2,6-diamino-4-pyrimidinyl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid

The title compound was prepared according to the same procedure as Example 1 (Yield of two steps 16.0%).

¹H NMR(CD₃OD) δ 3.61(1H, d, 14 Hz), 3.78(1H, d, 14 Hz), 3.80(2H, s), 3.98(2H, m), 5.10(1H, d, 5.2 Hz), 5.68((1H, d, 5.3 Hz), 6.01(1H, s), 6.20(1H, m), 7.20(2H, m), 7.35(1H, m), 7.52(1H, m)

Mass(m/e) 598

### Example 4

### Synthesis of 1,4-diamino-2-({(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}sulfanyl)pyrimidin-1-ium

The title compound was prepared according to the same procedure as Example 1 (Yield of two steps 12.5%).

¹H NMR(DMSO) δ 3.40(1H, d, 15 Hz), 3.75(1H, d, 14.9 Hz), 3.77(2H, s), 3.95(2H, s), 5.01(1H, s), 5.48(3H, m), 6.55(1H, m), 6.66(1H, m), 7.42(6H, m), 8.01(1H, s), 9.20(1H, s)

Mass(m/e) 599

### Preparation 2

### Synthesis of 2-(2,5-dichloroanilino)acetic acid

2,5-Dichloroaniline(10 g) and glyoxylic acid(6.2 g) were dissolved in methanol(100 ml), which was then cooled to 0° C. and stirred for 40 minutes. Sodium cyanoborohydride (4.5 g) was slowly added dropwise thereto and the resulting mixture was stirred for about 3 hours at room temperature. The solvent was removed under reduced pressure and excess diethylether was added to the residue. The organic layer was washed with diluted hydrochloric acid solution and water, dried over magnesium sulfate, and filtered. The filtrate was distilled under reduced pressure and the residue was solidified using hexane to give the title compound (Yield 60%).

¹H NMR(CDCl₃) δ 3.92(2H, d, 5.5 Hz), 5.86(1H, m), 6.66(2H, m), 7.26(1H, m)

Mass(m/e) 219

### Preparation 3

### Synthesis of 4-methoxybenzyl (6R,7R)-3-[(E)-3-chloro-1-propenyl]-7-{[2-(2,5-dichloroanilino)acetyl]amino}-8-oxo-5-thia-1-azabicyclo[4.2.0]oct-2-ene-2-carboxylate

The title compound was prepared according to the same procedure as Preparation 1 (Yield of two steps 75.5%).

¹H NMR(CDCl₃) δ 3.24(1H, d, 18.3 Hz), 3.44(1H, d, 18 Hz), 3.72(1H, dd, 7.8 Hz, 11.5 Hz), 3.79(3H, s), 3.86(2H, m), 3.97(1H, dd, 6.4 Hz, 14.6 Hz), 4.98(1H, m), 5.03(1H, d, 5 Hz), 5.11(2H, s), 5.22(1H, m), 5.71(1H, m), 5.81(1H, m), 6.21(1H, d, 11 Hz), 6.55(1H, d, 2.3 Hz), 6.73(1H, dd, 2.3 Hz, 8.3 Hz), 6.89(2H, m), 7.30(3H, m)

Mass(m/e) 595

### Example 5

### Synthesis of (6R,7R)-3-{(E)-3-[(2,6-diamino-4-pyrimidinyl)sulfanyl]-1-propenyl-7-{[2-(2,5-dichloroanilino)acetyl]amino}-8-oxo-5-thia-1-azabicyclo[4.2.0]oct-2-ene-2-carboxylic acid

The title compound was prepared according to the same procedure as Example 1 (Yield of two steps 18.0%).

¹H NMR(DMSO) δ 3.68(1H, d, 14 Hz), 3.81(1H, d, 14 Hz), 3.92(2H, s), 3.99(2H, m), 5.12(1H, d, 4.6 Hz), 5.66(1H, m), 5.98(1H, s), 6.02(1H, m), 6.15(1H, m), 6.50(1H, d, 2.3 Hz), 6.63(1H, dd, 2.3 Hz, 8.3 Hz), 6.92(1H, d, 14 Hz), 7.25(2H, d, 8.7 Hz), 8.05(2H, s), 9.10(1H, d, 8.3 Hz)

Mass(m/e) 581

### Preparation 4

### Synthesis of 4-methoxybenzyl (6R,7R)-3-(chloromethyl)-7-({2-[(2,5-dichlorophenyl)sulfanyl]acetyl}amino)-8-oxo-5-thia-1-azabicyclo[4.2.0]oct-2-ene-2-carboxylate

(6R,7R)-4-methoxybenzyl 7-amino-3-(chloromethyl)-8-oxo-5-thia-1-azabicyclo[4.2.0]oct-2-ene-2-carboxylate hydrochloride(1.5 g, 3.70 mmol) and 2,5-dichlorophenyl thioacetic acid(0.877 g, 3.70 mmol) were dissolved in dichloromethane(20 ml). Temperature in the reaction vessel was lowered to −30° C., and each of pyridine(0.75 ml, 9.25 mmol) and phosphoryloxy chloride(0.45 ml, 4.81 mmol) was slowly added dropwise thereto. The temperature in the reaction vessel was gradually raised to 0° C. during which the reaction mixture was stirred for 3 hours. The reaction mixture was diluted with excess ethyl acetate, washed with saturated ammonium chloride solution, 5% aqueous sodium bicarbonate solution and aqueous sodium chloride solution once per each solution, dried over anhydrous magnesium sulfate, and filtered. The filtrate was distilled under reduced pressure and the residue was purified by column chromatography to give 1.57 g (Yield 72.2%) of the title compound.

¹HNMR(CDCl₃) δ 7.33˜7.29(3H, q, dd), 7.21(1H, d), 7.13(1H, d), 6.89˜6.87(1H, dd), 5.77˜5.75(1H, dd, J=4.15 Hz), 5.21(2H, s), 4.93˜4.92(1H, d, J=5.0 Hz), 4.52˜4.50(1H, Abq, J=11.45 Hz), 4.40˜4,38(1H, Abq, J=11.95 Hz), 3.82(3H, s), 3.79˜3.66(2H, q), 3.60˜3.57(1H, Abq, J=18.3 Hz), 3.41˜3.38(1H, Abq, J=18.3 Hz)

Mass(m/e) 586

### Example 6

### Synthesis of 1,4-diamino-2-({[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]methyl}sulfanyl)pyrimidin-1-ium

4-Methoxybenzyl (6R,7R)-3-(chloromethyl)-7-({2-[(2,5-dichlorophenyl)sulfanyl]acetyl}amino)-8-oxo-5-thia-1-azabicyclo[4.2.0]oct-2-ene-2-carboxylate(0.5 g, 0.8511 mmol) was dissolved in dimethylfomamide(5 ml), 1,4-diamino-2(1H)-pyrimidinthione ½ sulfate(0.115 g, 0.809 mmol) was added thereto, and the mixture was heated to 40° C. for 30 minutes. After all of the reactants were dissolved, the resulting solution was stirred for 3 hours at room temperature. The reaction solution was diluted with excess ethyl acetate, washed three times with aqueous sodium chloride solution, dried over anhydrous magnesium sulfate, and filtered. The filtrate was distilled under reduced pressure, and then the residue was purified by dichloromethane and diethylether and dried under nitrogen atmosphere. Thus obtained solid (0.6 g) was deprotected by trifluoroacetic acid and anisole and then purified by high pressure preparative liquid chromatography to give the title compound (Yield of two steps 25.5%).

¹HNMR(DMSO-d₆) δ 9.21˜9.19(1H, d, J=7.8 Hz), 7.49˜7.47(2H, m), 7.25(1H, d, J=8.25 Hz), 7.10(1H, d), 6.83˜6.81(1H, d, J=8.25 Hz), 5.57(1H, br, d), 5.00(1H, br, d), 4.62(1H, br, s), 3.92˜3.83(3H, s, m), 3.61˜3.39(2H, br, m)

Mass(m/e) 573

### Preparation 5

### Synthesis of 4-methoxybenzyl (6R,7R)-3-[(Z)-3-chloro-1-propenyl]-7-({2-[(2,6-dichloro-4-pyridinyl)sulfanyl]acetyl}amino)-8-oxo-5-thia-1-azabicyclo[4.2.0]oct-2-ene-2-carboxylate

4-Methoxybenzyl (6R,7R)-7-amino-3-[3-chloro-1-propenyl]-8-oxo-5-thia-1-azabicyclo[4.2.0]oct-2-ene-2-carboxylate hydrochloride(1.8 g, 4.22 mmol) and 2-[(2,6-dichloro-4-pyridinyl)sulfanyl]acetic acid(1.0 g, 4.22 mmol) were dissolved in dichloro- methane(20 ml). Temperature in the reaction vessel was lowered to −30° C., and each of pyridine(0.85 ml, 10.55 mmol) and phosphoryloxy chloride(0.51 ml,, 5.49 mmol) was slowly added dropwise thereto. The temperature in the reaction vessel was gradually raised to 0° C. during which the reaction mixture was stirred for 3 hours. The reaction mixture was diluted with excess ethyl acetate, washed with saturated ammonium chloride solution, 5% aqueous sodium bicarbonate solution and aqueous sodium chloride solution once per each solution, dried over anhydrous magnesium sulfate, and filtered. The filtrate was distilled under reduced pressure and the residue was purified by column chromatography to give 1.6 g (Yield 62.0%) of the title compound.

¹H NMR(DMSO) δ 9.31˜9.30(1H, d, J=8.25 Hz), 7.51(2H, s), 7.32˜7.31(2H, d, J=8.7 Hz), 6.93˜6.91(2H, d, J=8.7 Hz), 6.30˜6.27(1H, d, J=10.95 Hz), 5.74˜5.69(2H, m), 5.25˜5.06(3H, m), 4.11(1H, m), 4.01(2H, m), 3.95(1H, m), 3.76(3H, s), 3.68˜3.64(1H, m), 3.51˜3.47(1H, m)

Mass(m/e) 613

### Example 7

### Synthesis of (6R,7R)-7-({2-[(2,6-dichloro-4-pyridinyl)sulfanyl]acetyl}amino)-8-oxo-3-[(E)-3-(1H-pyrazolo[3,4-d]pyrimidin-4-ylsulfanyl]-1-propenyl]-5-thia-1-azabicyclo [4.2.0]oct-2-ene-2-carboxylic acid

4-Methoxybenzyl (6R,7R)-3-[(Z)-3-chloro-1-propenyl]-7-({2-[(2,6-dichloro-4-pyridinyl)sulfanyl]acetyl}amino)-8-oxo-5-thia-1-azabicyclo[4.2.0]oct-2-ene-2-carboxylate (0.38 g, 0.62 mmol) was dissolved in acetone(4 ml) and sodium iodide(0.17 g, 1.14 mmol) was added thereto. The reaction mixture was stirred for 1 hour at room temperature and distilled under reduced pressure. The residue was dissolved in ethyl acetate and washed with water and aqueous sodium chloride solution. The organic layer was dried over anhydrous magnesium sulfate and filtered, and the filtrate was distilled under reduced pressure. The residue was dissolved in dimethylformamide, 4-mercapto-1H-pyrazolo[3,4-d]-pyrimidine(0.096 g, 0.63 mmol) was added thereto, and the mixture was stirred for 24 hours at room temperature. The reaction mixture was diluted with excess ethyl acetate, water was added thereto, and the resulting solid was filtered. The filtrate was washed with water and aqueous sodium chloride solution, dried over anhydrous magnesium sulfate and filtered. The filtrate was distilled under reduced pressure. The residue was dissolved in a small amount of methylene chloride, purified by diethylether and filtered. The solid obtained by each method was dried under nitrogen atmosphere.

Thus obtained solid(70 mg) was deprotected by trifluoroacetic acid, anisole and triethylsilane, and then purified by high pressure preparative liquid chromatography to give 20 mg(Yield of two steps 5.3%) of the title compound.

¹H NMR(DMSO, 500 MHz) δ 9.21(1H, d, J=8.3 Hz, NH), 8.72(1H, s), 8.25(1H, s), 7.51(2H, s), 7.10(1H, d, J=16.0 Hz), 5.68˜5.73(1H, m), 4.92(1H, d, J=4.6 Hz), 4.12˜4.14(2H, m), 3.95˜4.03(2H, m), 2.88(1H, s), 2.72(1H, s)

Mass(m/e) 609

### Example 8

### Synthesis of (6R,7R)-3-{(E)-3-[(4,6-diamino-2-pyrimidinyl)sulfanyl]-1-propenyl}-7-({2-[(2,6-dichloro-4-pyridinyl)sulfanyl]acetyl}amino)-8-oxo-5-thia-1-azabicyclo [4.2.0]oct-2-ene-2-carboxylic acid

The title compound was prepared according to the same procedure as Example 7 (Yield of two steps 0.2%).

¹H NMR(D₂O, 500 MHz) δ 7.33(2H, s), 6.7(1H, d, J=16.0 Hz), 5.93(1H, m), 5.54(1H, d, J=4.6 Hz), 5.43(1H, s), 5.04(1H, d, J=4.6 Hz), 4.72(2H, s), 3.76(2H, d, J=6.9 Hz), 3.46˜3.56(2H, m)

Mass(m/e) 599

### Example 9

### Synthesis of (6R,7R)-3-{(E)-3-[(4-amino-1H-pyrazolo[3,4-d]pyrimidin-6-yl)sulfanyl]-1-propenyl}-7-({2-[(2,6-dichloro-4-pyridinyl)sulfanyl]acetyl}amino)-8-oxo-5-thia-1-azabicyclo[4.2.0]oct-2-ene-2-carboxylic acid

4-Methoxybenzyl (6R,7R)-3-[(Z)-3-chloro-1-propenyl]-7-({2-[(2,6-dichloro-4-pyridinyl)sulfanyl]acetyl}amino)-8-oxo-5-thia-1-azabicyclo[4.2.0]oct-2-ene-2-carboxylate (0.15 g, 0.24 mmol) was dissolved in dimethylformamide(1.5 ml) and sodium iodide(0.073 g, 0.49 mmol) was added thereto. The reaction mixture was stirred for 1 hour at room temperature, 4-amino-1H-pyrazolo[3,4-d]pyrimidin-6-thiol(0.053 g, 0.32 mmol) was added, and the resulting mixture was stirred for 24 hours at room temperature. The reaction mixture was diluted with excess ethyl acetate, water was added, and the resulting solid was filtered. The filtrate was washed with water and aqueous sodium chloride solution, dried over anhydrous magnesium sulfate and filtered. The filtrate was distilled under reduced pressure, and then the residue was dissolved in a small amount of methylene chloride, purified by diethylether and filtered. The solid obtained by each method was dried under nitrogen atmosphere.

Thus obtained solid(30 mg) was deprotected by trifluoroacetic acid, anisole and triethylsilane, and then purified by high pressure preparative liquid chromatography to give 2.10 mg(Yield of two steps 1.4%) of the title compound.

¹H NMR(D₂O, 400 MHz) δ 7.78(1H, s), 7.00(2H, s), 6.70(1H, d, J=14.8 Hz), 5.62(1H, m), 5.42(1H, m), 4.98(1H, m), 3.67˜3.75(2H, m), 3.45˜3.53(2H, m), 3.22˜3.36(2H, m)

Mass(m/e) 624

### Example 10

### Synthesis of (6R,7R)-3-{(E)-3-[(2-amino-6-hydroxy-4-pyrimidinyl)sulfanyl]-1-propenyl}-7-({2- [(2,6-dichloro-4-pyridinyl)sulfanyl]acetyl}amino)-8-oxo-5-thia-1-azabicyclo[4.2.0]oct-2-ene-2-carboxylic acid

The title compound was prepared according to the same procedure as Example 9 (Yield of two steps 12.9%).

¹H NMR(D₂O, 400 MHz) δ 7.19(2H, s), 6.60(1H, d, J=15.6 Hz), 5.51˜5.60(1H, m), 5.48(1H, s), 5.44(1H, d, J=4.4 Hz), 5.01(1H, d, J=4.4 Hz), 3.82˜3.87(2H, m), 3.55˜3.65(2H, m), 3.16˜3.38(2H, m),

Mass(m/e) 600

### Example 11

### Synthesis of (6R,7R)-3-{(E)-3-[(2,6-diamino-4-pyrimidinyl)sulfanyl]-1-propenyl}-7-({2-[(2,6-dichloro-4-pyridinyl)sulfanyl]acetyl}amino)-8-oxo-5-thia-1-azabicyclo[4.2.0]oct-2-ene-2-carboxylic acid

The title compound was prepared according to the same procedure as Example 9 (Yield of two steps 15.7%).

¹H NMR(DMSO, 400 MHz) δ 9.30(1H, d, J=8.0 Hz, NH), 7.52(2H, s), 6.92(1H, d, J=16 Hz), 6.19(2H, brs), 6.00˜6.09(1H, m), 5.93(2H, brs), 5.60(2H, m), 5.08(1H, d, J=4 Hz), 4.00˜4.05(2H, m), 3.57˜3.85(4H, m)

Mass(m/e) 599

### Example 12

### Synthesis of (6R,7R)-7-({2-[(2,6-dichloro-4-pyridinyl)sulfanyl]acetyl}amino)-3-((E)-3-{[2-(ethylsulfanyl)-6-methyl-4-pyrimidinyl]sulfanyl}-1-propenyl)-8-oxo-5-thia-1-azabicyclo[4.2.0]oct-2-ene-2-carboxylic acid

The title compound was prepared according to the same procedure as Example 9 (Yield of two steps 7.0%).

¹H NMR(D₂O, 400 MHz) δ 7.18(2H, s), 6.74(1H, s), 6.02(1H, d, J=11.2 Hz), 5.56˜5.60(1H, m), 5.41˜5.42(1H, m), 4.98˜5.00(1H, m), 3.74˜3.77(2H, m), 3.50˜3.63(2H, m), 3.30˜3.43(2H, m), 2.90˜2.98(2H, m), 2.15(3H, s), 1.16(3H, t)

Mass(m/e) 643

### Example 13

### Synthesis of 7-amino-5-({(E)-3-[(6R,7R)-2-carboxy-7-({2-[(2,6-dichloro-4-pyridinyl)sulfanyl]acetyl}amino)-8-oxo-5-thia-1-azabicyclo[4.2.0]oct-2-en-3-yl]-2-propenyl}sulfanyl)-1H-[1,2,4]triazolo[1,5-c]pyrimidin-4-ium

The title compound was prepared according to the same procedure as Example 9 (Yield of two steps 22.5%).

¹H NMR(D₂O, 400 MHz) δ 7.98(1H, s), 7.11(2H, s), 6.78(1H, d, J=15.2 Hz), 6.12(1H, s), 5.63˜5.69(1H, m), 5.43(1H, d, J=4.8 Hz), 5.00(1H, d, J=4.8 Hz), 3.86˜3.91(2H, m), 3.69˜3.75(2H, m), 3.19˜3.35(2H, m)

Mass(m/e) 625

### Example 14

### Synthesis of 2,7-diamino-5-({(E)-3-[(6R,7R)-2-carboxy-7-({2-[(2,6-dichloro-4-pyridinyl)sulfanyl]acetyl}amino)-8-oxo-5-thia-1-azabicyclo[4.2.0]oct-2-en-3-yl]-2-propenyl]sulfanyl)-1-methyl-1H-[1,2,4]triazolo[1,5-c]pyrimidin-4-ium

The title compound was prepared according to the same procedure as Example 9 (Yield of two steps 2.2%).

¹H NMR(DMSO, 400 MHz) δ 9.25(1H, d, J=8.0 Hz, NH), 7.70(2H, s), 7.53(2H, s), 7.40(1H, d, J=15.6 Hz), 6.16(1H, s), 5.55˜5.59(1H, m), 5.45˜5.51(1H, m), 5.01(1H, m), 3.97˜4.02(2H, m), 3.67˜3.74(2H, m)

Mass(m/e) 654

### Example 15

### Synthesis of (6R,7R)-7-({2-[(2,6-dichloro-4-pyridinyl)sulfanyl]acetyl}amino)-3-[(E)-3-({4-hydroxy-6-[(2-hydroxyethyl)amino]-2-pyrimidinyl]sulfanyl}-1-propenyl]-8-oxo-5-thia-1-azabicyclo[4.2.0]oct-2-ene-2-carboxylic acid

The title compound was prepared according to the same procedure as Example 9 (Yield of two steps 1.6%).

¹H NMR(D₂O, 400 MHz) δ 7.37(1H, s), 6.76(1H, d, J=15.6 Hz), 5.92˜6.02(1H, m), 5.56(1H, d, J=4.4 Hz), 5.14(1H, s), 5.08(1H, d, J=4.4 Hz), 4.02(2H, m), 3.91(1H, Abq, J=6.8 Hz), 3.71˜3.75(2H, m), 3.54(1H, Abq, J=7.2 Hz), 3.35˜3.40(2H, m)

Mass(m/e) 644

### Example 16

### Synthesis of 4,6-diamino-2-({(E)-3-[(6R,7R)-2-carboxy-7-({2-[(2,6-dichloro-4-pyridinyl)sulfanyl]acetyl}amino)-8-oxo-5-thia-1-azabicyclo[4.2.0]oct-2-en-3-yl]-2-propenyl}sulfanyl)-1-ethylpyrimidin-1-ium

4-Methoxybenzyl (6R,7R)-3-[(Z)-3-chloro-1-propenyl]-7-({2-[(2,6-dichloro-4-pyridinyl)sulfanyl]acetyl}amino)-8-oxo-5-thia-1-azabicyclo[4.2.0]oct-2-ene-2-carboxylate (0.4 g, 0.689 mmol) was dissolved in acetone(5 ml) and sodium iodide(0.3 g, 2.001 mmol) was added thereto. The reaction mixture was stirred for 1 hour at room temperature and the solvent was removed by distillation under reduced pressure. The residue was dissolved in dimethylformamide(5 ml), 4,6-diamino-1-ethyl-2(1H)-pyrimidinthione(0.136 g,0.803 mmol) was added, and the resulting mixture was stirred for 3 hours at room temperature. The reaction mixture was diluted with excess ethyl acetate, washed twice with water, dried over anhydrous magnesium sulfate and filtered. The filtrate was distilled under reduced pressure, and then the residue was purified by diethylether and dried under nitrogen atmosphere.

Thus obtained solid(0.35 g) was deprotected by trifluoroacetic acid, anisole and triethylsilane, and then purified by high pressure preparative liquid chromatography to give 0.017 g(Yield of two steps 4.1%) of the title compound.

¹HNMR(DMSO-d6) δ 9.23˜9.21(1H, d, J=8.25 Hz), 7.89(1H, br, s), 7.52(2H, s), 7.32˜7.35(1H, d, J=15.58 Hz), 5.51(1H, s), 5.46˜5.45(1H, d, J=5.04 Hz), 4.97˜4.96(1H, d, J=5.04 Hz), 4.00˜3.96(3H, m), 3.85˜3.84(1H, m), 3.40˜3.34(4H, m), 1.22(3H, t)

Mass(m/e) 628

### Example 17

### Synthesis of 1,2-diamino-4-({(E)-3-[(6R,7R)-2-carboxy-7-({2-[(2,6-dichloro-4-pyridinyl)sulfanyl]acetyl}amino)-8-oxo-5-thia-1-azabicyclo[4.2.0]oct-2-en-3-yl]-2-propenyl}sulfanyl)-6,7-dihydro-5H-cyclopenta[d]pyrimidin-1-ium

The title compound was prepared according to the same procedure as Example 16 (Yield of two steps 6.2%).

¹HNMR(DMSO-d₆) δ 9.22˜9.20(1H, d, J=7.8 Hz), 7.51(2H, s), 7.41˜7.39(1H, d, J=15.58 Hz), 6.38(1H, br, m), 6.29(1H, br, s), 5.49˜5.46(2H, br, m), 4.98˜4.97(1H, d, J=5.05 Hz), 4.01˜3.95(3H, q, m), 3.80(1H, m), 3.65(1H, Abq, J=16.5 Hz), 3.09˜3.07(2H, m), 2.71˜2.70(2H, m), 2.11˜2.10(2H, m)

Mass(m/e) 640

### Example 18

### Synthesis of 4,6-diamino-1-({(E)-3-[(6R,7R)-2-carboxy-7-({2-[(2,6-dichloro-4-pyridinyl)sulfanyl]acetyl}amino)-8-oxo-5-thia-1-azabicyclo[4.2.0]oct-2-en-3-yl]-2-propenyl}pyrimidin-1-ium

4-Methoxybenzyl (6R,7R)-3-[(Z)-3-chloro-1-propenyl]-7-({2-[(2,6-dichloro-4-pyridinyl)sulfanyl]acetyl}amino)-8-oxo-5-thia-1-azabicyclo[4.2.0]oct-2-ene-2-carboxylate (0.4 g, 0.689 mmol) was dissolved in acetone(5 ml) and sodium iodide(0.3 g, 2.001 mmol) was added thereto. The reaction mixture was stirred for 1 hour at room temperature and the solvent was removed by distillation under reduced pressure. The residue was dissolved in dimethylformamide(5 ml), 1,2-dihydro-4,6-pyrimidinediamine(0.088 g, 0.803 mmol) was added, and the resulting mixture was stirred for 24 hours at room temperature. The reaction mixture was diluted with excess ethyl acetate, washed twice with water, dried over anhydrous magnesium sulfate and filtered. The filtrate was distilled under reduced pressure, and then the residue was purified by diethylether and dried under nitrogen atmosphere.

Thus obtained solid(0.20 g) was deprotected by trifluoroacetic, acid, anisole and triethylsilane, and then purified by high pressure preparative liquid chromatography to give 0.017 g(Yield of two steps 4.5%) of the title compound.

¹HNMR(DMSO-d₆) δ 9.27(1H, br, s), 8.28(1H, s), 7.92(1H, br, s), 7.60(1H, br, s), 7.47(2H, s), 7.05˜7.04(1H, dd, 5.5 Hz), 5.65(1H, s) 5.45(1H, br, m), 4.94˜4.93(1H, d, J=4.1 Hz), 4.67(1H, br, s), 4.02˜3.97(2H, q, J=15.6 Hz), 3.42˜3.40(2H, m)

Mass(m/e) 568

### Example 19

### Synthesis of (6R,7R)-7-amino-5-({(E)-3-[2-carboxy-7-({2-[(2,5-dichlorophenyl)sulfanyl]acetyl]amino)-8-oxo-5-thia-1-azabicyclo[4.2.0]oct-2-en-3-yl]-2-propenyl}sulfanyl)-3H-[1,2,4]triazolo[1,5-c]pyrimidin-4-ium

The title compound was prepared according to the same procedure as Example 1 (Yield of two steps 5.7%).

¹H NMR(DMSO) δ 3.33(2H, br, m), 3.87˜3.98(4H, br, m), 4.96˜4.97(1H, d, 4.55 Hz), 5.45˜5.47(1H, dd, 4.1 Hz, 8.7 Hz), 5.59(1H, m), 6.12(1H, br, s), 6.96(1H, br, s), 7.24˜7.25(1H, br, m), 7.37(1H, br, d), 7.47˜7.49(2H, br, m), 8.12(1H, s), 9.16˜9.17(1H, d, 7.75 Hz)

Mass(m/e) 624

### Example 20

### Synthesis of (6R,7R)-3-{(E)-3-[(4-amino-6,7-dihydro-5H-cyclopenta[d]pyrimidin-2-yl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid

The title compound was prepared according to the same procedure as Example 1 (Yield of two steps 4.9%).

¹H NMR(DMSO) δ 1.69(4H, br, m), 2.24(2H, br, m), 3.68˜3.72(2H, br, m), 3.93(2H, br, s), 4.96(1H, d, 4.45 Hz), 5.47(1H, dd, 4.4 Hz, 8.7 Hz), 5.64(1H, m), 6.70(2H, br, m), 7.08˜7.11(1H, br, d), 7.25(1H, br, m), 7.49(2H, br, m), 9.19(1H, d, 7.80 Hz)

Mass(m/e) 623

### Example 21

### Synthesis of (6R,7R)-1,2-diamino-4-({(E)-3-[2-carboxy-7-({2-[(2,5-dichlorophenyl)sulfanyl]acetyl}amino-8-oxo-5-thia-1-azabicyclo[4.2.0]oct-2-en-3-yl-2-propenyl}sulfanyl)-6,7-dihydro-5H-cyclopenta[d]pyrimidin-1-ium

The title compound was prepared according to the same procedure as Example 1 (Yield of two steps 1.8%).

¹H NMR(DMSO) δ 2.06˜2.09(2H, m), 2.72(2H, m), 3.04(2H, m), 3.72˜3.75(2H, m), 3.9(2H, s), 4.96˜4.97(1H, d, 5.04 Hz), 5.46(2H, m), 6.43(2H, br, s), 7.25(1H, d), 7.39(1H, d, 15.12 Hz), 7.48(2H, m), 8.36(1H, s), 9.14˜9.15(1H, d, 7.79 Hz), 9.77(1H, s)

Mass(m/e) 639

### Example 22

### Synthesis of (6R,7R)-2,6-diamino-4-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}sulfanyl)-1-methylpyrimidin-1-ium

The title compound was prepared according to the same procedure as Example 1 (Yield of two steps 1.5%).

¹H NMR(DMSO) δ 3.53(1H, ABq, 18.3 Hz), 3.91˜3.97(5H, m, s), 4.97(1H, d, 4.6 Hz), 5.50˜5.57(2H, dd, m, 5.05 Hz,8.25 Hz), 6.36(1H, s), 7.03˜7.06(1H, m), 7.24˜7.25(1H, d, 8.25 Hz), 7.46˜7.49(2H, m), 7.69(1H,s), 7.87(1H, br, s), 8.82(1H, br, s), 9.15˜9.17(1H, d, 8.2 Hz)

Mass(m/e) 613

### Example 23

### Synthesis of (6R,7R)-4,6-diamino-2-({(E)-3-[2-carboxy-7-({2-[(2,5-dichlorophenyl) sulfanyl]acetyl}amino)-8-oxo-5-thia-1-azabicyclo[4.2.0]oct-2-en-3-yl]-2-propenyl}sulfanyl)-1-methyl-5-[(methylamino)methyl]pyrimidin-1-ium

The title compound was prepared according to the same procedure as Example 1 (Yield of two steps 2.5%).

¹H NMR(DMSO) δ 2.32(3H, s), 3.42˜3.49(3H, m), 3.91(4H, s, m), 4.96˜4.97(1H, d, 5.05 Hz), 5.45˜5.47(2H, m), 7.25(1H, d, 8.25 Hz), 7.46˜7.49(2H, m), 9.16˜9.18(1H, d, 7.75 Hz)

Mass(m/e) 656

### Example 24

### Synthesis of (6R,7R)-3-{(E)-3-[(4,6-diamino-2-pyrimidinyl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid

The title compound was prepared according to the same procedure as Example 1 (Yield of two steps 1.8%).

¹H NMR(D₂O) δ 7.44˜7.27(3H, m), 6.72(1H, d, 7.8 Hz), 5.98˜5.91(1H, m), 5.50(1H, m), 5.35(1H, s), 5.0˜4.9(1H, m), 4.9˜4.8(2H, m), 3.77˜3.65(2H, m), 3.52˜3.44(2H, m)

Mass(m/e) 598

### Example 25

### Synthesis of (6R,7R)-3-{(E)-3-[(5,6-diamino-4-pyrimidinyl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid

The title compound was prepared according to the same procedure as Example 1 (Yield of two steps 1.0%).

¹H NMR(D₂O) δ 7.60˜7.25(2H, m), 7.14˜7.11(1H, m), 6.85(1H, d, 15.6 Hz), 5.83˜5.78(1H, m), 5.53(1H, d, 4.5 Hz), 4.94(1H, d, 4.8 Hz), 4.78(1H, s), 4.69˜4.67(2H, m), 3.84˜3.69(2H, m), 3.54˜3.46(2H, m)

Mass(m/e) 598

### Example 26

### Synthesis of (6R,7R)-3-{(E)-3-[(4,6-diamino-5-methyl-2-pyrimidinyl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid

The title compound was prepared according to the same procedure as Example 1 (Yield of two steps 1.2%).

¹H NMR(D₂O) δ 7.66˜7.11(3H, m), 6.97(1H, d, 15.3 Hz), 5.85˜5.75(1H, m), 5.53(1H, d, 4.7 Hz), 4.95(1H, d, 4.8 Hz), 4.70˜4.60(2H, m), 3.73˜3.47(4H, m), 1.77(3H, s)

Mass(m/e) 612

### Example 27

### Synthesis of (6R,7R)-7-({2-[(2,5-dichlorophenyl)sulfanyl]acetyl}amino)-8-oxo-3-[(E)-3-(1H-pyrazolo[3,4-di pyrimidin-4-ylsulfanyl)-1-propenyl-5-thia-1-azabicyclo[4.2.0]oct-2-ene-2-carboxylic acid

The title compound was prepared according to the same procedure as Example 1 (Yield of two steps 1.2%).

¹H NMR(D₂O) δ 8.47(1H, s), 8.01(1H, s), 7.18˜7.12(2H, m), 6.97˜6.96(2H, m), 6.90˜6.86(1H, m), 5.90˜5.89(1H, m), 5.55˜5.54(1H, m), 5.00˜4.92(1H, m), 4.82˜4.76(2H, m), 3.85˜3.67(2H, m), 3.46˜3.36(2H, m)

Mass(m/e) 608

### Example 28

### Synthesis of (6R,7R)-7-({2-[(2,5-dichlorophenyl)sulfanyl]acetyl}amino)-3-((E)-3-{[6-methyl-2-(methylsulfanyl)-4-pyrimidinyl]sulfanyl}-1-propenyl)-8-oxo-5-thia-1-azabicyclo[4.2.0]oct-2-ene-2-carboxylic acid

The title compound was prepared according to the same procedure as Example 1 (Yield of two steps 1.1%).

¹H NMR(DMSO-d₆) δ 9.18(1H, d, 8.2 Hz), 7.49˜7.47(2H, m), 7.46˜7.40(1H, m), 7.07(1H, d, 16 Hz), 6.99(1H, s), 5.69˜5.63(1H, m), 5.45(1H, dd, 8.0 Hz, 4.8 Hz), 4.95(1H, d, 4.6 Hz), 3.92(1H, s), 3.91˜3.89(2H, m), 3.41˜3.33(4H, m), 2.50(3H, s), 2.30(3H, s)

Mass(m/e) 628

### Example 29

### Synthesis of (6R,7R)-4,6-diamino-2-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}sulfanyl)-1-ethylpyrimidin-1-ium

The title compound was prepared according to the same procedure as Example 1 (Yield of two steps 4.7%).

¹H NMR(DMSO-d₆) δ 9.16(1H, brs), 7.83(1H, brs), 7.49˜7.46(2H, m), 7.35(1H, m), 7.25(1H, m), 5.51(1H, s), 5.51˜5.46(1H, m), 4.96(1H, d, 4.5 Hz), 3.99˜3.95(2H, m), 3.92(2H, q), 3.90˜3.85(2H, m), 3.40˜3.32(2H, m), 1.23(3H, t, 7.3 Hz)

Mass(m/e) 627

### Example 30

### Synthesis of (6R,7R)-3-((E)-3-{[4-amino-6-(methylamino)-2-pyrimidinyl]sulfanyl}-1-propenyl)-7-({2-[(2,5-dichlorophenyl)sulfanyl]acetyl}amino)-8-oxo-5-thia-1-azabicyclo[4.2.0]oct-2-ene-2-carboxylic acid

The title compound was prepared according to the same procedure as Example 1 (Yield of two steps 5.1%).

Mass(m/e) 612

### Example 31

### Synthesis of (6R,7R)-4,6-diamino-1-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}5-methylpyrimidin-1-ium

The title compound was prepared according to the same procedure as Example 1 (Yield of two steps 1.0%).

¹H NMR(DMSO-d₆) δ 9.21(1H, d, 8.2 Hz), 8.30(1H, d, 11.0 Hz), 7.64(1H, brs), 7.49˜7.46(2H, m), 7.30˜7.23(1H, m), 7.08(1H, d, 15.6 Hz), 5.65˜5.57(1H, m), 5.47˜5.44(1H, m), 4.95(1H, s), 4.74(1H, d, 5.0 Hz), 4.01˜3.78(4H, m), 3.47˜3.35(2H, m), 1.88(3H, s)

Mass(m/e) 581

### Example 32

### Synthesis of (6R,7R)-2,7-diamino-6-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}[1,2,4]triazolo[1,5-c]pyrimidin-6-ium

The title compound was prepared according to the same procedure as Example 1 (Yield of two steps 2.8%).

¹H NMR(DMSO-d₆) δ 9.21(1H, d, 7.8 Hz), 8.3(1H, s), 7.94(1H, s), 7.53˜7.42(2H, m), 7.32˜7.21(1H, m), 7.08(1H, d, 16 Hz), 5.69(1H, s), 5.68˜5.60(1H, m), 5.48˜5.46(1H, m), 4.96˜4.95(1H, m), 4.69(1H, s), 3.92˜3.82(4H, m), 3.45˜3.35(2H, m)

Mass(m/e) 607

### Example 33

### Synthesis of (6R,7R)-4-amino-1-({(E)-3-[2-carboxy-7-({2-[(2,5-dichlorophenyl)sulfanyl]acetyl}amino)-8-oxo-5-thia-1-azabicyclo[4.2.0]oct-2-en-3-yl] -2-propenyl}-2-methylpyrimidin-1-ium

The title compound was prepared according to the same procedure as Example 1 (Yield of two steps 1.3%).

¹H NMR(DMSO-d₆) δ 9.10(1H, d, 7.5 Hz), 8.05(1H, d, 7.3 Hz), 8.04˜7.34(2H, m), 7.15˜7.11(1H, m), 6.95(1H, d, 15.6 Hz), 6.65(1H, d, 7.26 Hz), 5.68˜5.57(1H, m), 5.38˜5.34(1H, m), 4.84(1H, d, 4.8 Hz), 4.68(1H, d, 5.5 Hz), 3.86˜3.67(4H, m), 3.50˜3.31(2H, m), 2.39(3H, s)

Mass(m/e) 566

### Example 34

### Synthesis of (6R,7R)-4-amino-1-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-6,7-dihydro-5H-cyclopenta[d]pyrimidin-1-ium

The title compound was prepared according to the same procedure as Example 1 (Yield of two steps 1.0%).

¹H NMR(DMSO-d₆) δ 9.52(1H, brs), 8.68(1H, brs), 7.52˜7.42(2H, m), 7.25˜7.21(1H, m), 7.06˜7.04(1H, m), 5.78˜5.65(1H, m), 5.47˜5.41(1H, m), 4.97˜4.90(1H, m), 4.79(1H, s), 3.91˜3.61(4H, m), 3.46˜3.30(2H, m), 2.74˜2.60(4H, m), 1.89˜1.74(2H, m)

Mass(m/e) 592

### Example 35

### Synthesis of (6R,7R)-4,5,6-triamino-1-({(E)-3-[2-carboxy-7-({2-[(2,5-dichlorophenyl)sulfanyl]acetyl}amino)-8-oxo-5-thia-1-azabicyclo[4.2.0]oct-2-en-3-yl] -2-propenyl}pyrimidin-1-ium

The title compound was prepared according to the same procedure as Example 1 (Yield of two steps 3.2%).

¹H NMR(DMSO-d₆) δ 9.17(1H, m), 7.50˜7.43(2H, m), 7.25˜7.23(1H, m), 6.91(1H, d, 16.0 Hz), 5.97(1H, s), 5.80(1H, s), 5.79˜5.75(1H, m), 4.95(1H, d, 4.6 Hz), 3.95˜3.92(2H, m), 3.51˜3.35(4H, m)

Mass(m/e) 582

### Example 36

### Synthesis of (6R,7R)-4,6-diamino-1-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}pyrimidin-1-ium

The title compound was prepared according to the same procedure as Example 1 (Yield of two steps 2.0%).

¹H NMR(DMSO-d₆) δ 9.20(1H, brs), 8.31(1H, s), 7.55˜7.43(2H, m), 7.25˜7.21(1H, m), 7.05(1H, d), 5.69(1H, s), 5.65˜5.59(1H, m), 5.47˜5.43(1H, m), 5.02˜4.92(1H, m), 4.72˜4.63(2H, m), 4.00˜3.92(2H, m), 3.52˜3.40(2H, m)

Mass(m/e) 567

### Example 37

### Synthesis of 4-amino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-6-(dimethylamino)-2-methylpyrimidin-1-ium

The title compound was prepared according to the same procedure as Example 1 (Yield of two steps 5.5%).

¹H NMR(DMSO) δ 2.26(3H, s), 2.80(6H, s), 3.19(2H, m), 3.42(2H, m), 3.78˜3.86(2H, m), 4.98˜5.04(1H,d, J=4.8 Hz), 5.53(2H, m), 5.77˜5.81(1H, m), 6.58˜6.61(1H,d, J=15.1 Hz), 6.97˜6.98(1H, m), 7.14˜7.24(2H, m)

Mass(m/e) 609

### Example 38

### Synthesis of 4-amino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl] -2-propenyl}-2-methyl-6-(methylamino)pyrimidin-1-ium

The title compound was prepared according to the same procedure as Example 1 (Yield of two steps 3.4%).

¹H NMR(D₂O) δ 2.28(3H, s), 2.84(3H, s), 3.16(2H, d), 3.48˜3.51(2H, m), 3.80˜3.91(2H, m), 4.99˜5.00(1H,d, J=4.6 Hz), 5.50(1H, m), 5.83(1H, m), 6.43˜6.46(1H, d, J=15.1 Hz), 7.22(11H, m), 7.39(1H, ,m)

Mass(m/e) 595

### Example 39

### Synthesis of 4-amino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-2-methylpyrimidin-1-ium

The title compound was prepared according to the same procedure as Example 1 (Yield of two steps 1.3%).

¹H NMR(DMSO, 300 MHz) δ 9.10(2H, d, J=7.6 Hz, NH), 8.05(1H, d, J=7.3 Hz), 7.34˜7.42(2H, m), 7.11˜7.15(1H, m), 6.95(1H, d, J=15.6 Hz), 6.65(1H, d, J=7.2 Hz), 5.57˜5.63(1H, m), 5.34˜5.38(1H, m), 4.85(1H, d, J=4.89 Hz), 4.68(1H, d, J=5.52 Hz), 3.67˜3.86(4H, m), 2.39(3H, s)

Mass(m/e) 567

### Example 40

### Synthesis of 4,6-diamino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-5-methylpyrimidin-1-ium

The title compound was prepared according to the same procedure as Example 1 (Yield of two steps 0.2%).

¹H NMR(DMSO, 500 MHz) δ 9.21(1H, d, J=8.25 Hz, NH), 8.30(1H, s), 7.46˜7.49(2H, m), 7.23˜7.27(1H, m), 7.09(1H, d, J=15.6 Hz), 5.56˜5.64(1H, m), 5.44˜5.47(1H, m), 4.94˜4.97(1H, m), 4.74˜4.76(2H, m), 3.89˜3.98(4H, m), 1.88(3H, s)

Mass(m/e) 582

### Example 41

### Synthesis of 4-amino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-2-methyl-6-(methylamino)pyrimidin-1-ium

The title compound was prepared according to the same procedure as Example 1 (Yield of two steps 3.4%).

¹H NMR(D₂O, 500 MHz) δ 7.20˜7.42(3H, m), 6.45(1H, d, J=15.1 Hz), 5.80˜5.86(1H, m), 5.47˜5.53(1H, m), 5.00(1H, d, J=4.6 Hz), 3.80˜3.91(2H, m), 3.45˜3.53(2H, m), 3.12˜3.18(2H, m), 2.84(3H, s), 2.28(3H, s)

Mass(m/e) 596

### Experiment 1

### Minimum Inhibitory Concentration (MIC)

The effectiveness of the compound according to the present invention was determined by obtaining Minimum Inhibitory Concentration (MIC) of the compounds prepared by the above examples (Compounds I-1 to I-41) and vancomycin, which is the known compound having a potent activity against gram-positive strains, as the control drug against the standard strains. Specifically, Minimum Inhibitory Concentration was obtained by diluting the test compound according to a double dilution method, dispersing them in Mueller-Hinton agar medium, inoculating each of the test strain having 10⁷ cfu (colony forming unit) per ml in an amount of 2 μl to the medium and then incubating them at 37° C. for 20 hours. The results are shown in the following Tables 1 and 2. From the result of Minimum Inhibitory Concentration test, it can be seen that the compound according to the present invention has a good activity against major pathogenic microorganisms, which cause hospital infection, including MRSA strains.

TABLE 1
Sensitivity test result using standard strains (μg/ml)

| | | S. | S. | S. | E. |
|---|---|---|---|---|---|
| | Staphylococcus | aureus | aureus | epidermidis | faecalis |
| | aureus giorgio | 77 | 241 | R005 | L239 |
| 1-1 | <0.008 | 0.25 | 4 | 0.13 | 0.25 |
| 1-2 | <0.008 | 0.25 | 4 | 0.25 | 1 |
| I-3 | <0.008 | 0.13 | 4 | 0.13 | 0.5 |
| I-4 | <0.008 | 0.13 | 1 | 0.13 | 0.5 |
| I-5 | 0.031 | 0.5 | 16 | 0.5 | 1 |
| I-6 | <0.008 | 0.031 | 4 | 0.063 | 4 |
| I-19 | <0.008 | 0.13 | 4 | 0.25 | 0.25 |
| I-20 | 0.063 | 2 | 32 | 2 | 2 |
| I-21 | <0.008 | 0.063 | 1 | 0.063 | 0.25 |
| I-22 | <0.008 | 0.063 | 1 | 0.063 | 0.25 |
| I-23 | 0.016 | 0.25 | 4 | 0.25 | 1 |
| I-24 | 0.016 | 0.25 | 4 | 0.13 | 0.5 |
| I-25 | 0.063 | 0.5 | 16 | 1 | 2 |
| I-26 | 0.13 | 1 | 16 | 2 | 2 |
| I-27 | <0.008 | 0.13 | 2 | 0.13 | 0.13 |
| I-28 | 0.016 | 0.25 | 32 | 0.25 | 0.5 |
| I-29 | <0.008 | 0.063 | 1 | 0.031 | 0.13 |
| I-30 | 0.016 | 0.5 | 16 | 0.5 | 2 |
| I-31 | 0.016 | 1 | 8 | 0.25 | 2 |
| I-32 | 0.016 | 0.5 | 4 | 0.25 | 1 |
| I-33 | <0.008 | 0.26 | 2 | 0.063 | 0.5 |
| I-34 | <0.008 | 0.5 | 4 | 0.13 | 1 |
| I-35 | 0.016 | 0.25 | 2 | 0.25 | 0.5 |
| I-36 | <0.008 | 0.13 | 1 | 0.063 | 0.25 |
| I-37 | 0.016 | 0.25 | 4 | 0.25 | 0.5 |
| I-38 | 0.031 | 1 | 8 | 0.5 | 1 |
| I-39 | <0.008 | 0.25 | 2 | 0.063 | 0.5 |
| I-40 | 0.016 | 1 | 8 | 0.25 | 2 |
| I-41 | 0.031 | 1 | 8 | 0.5 | 1 |
| Vancomycin | 1 | 1 | 2 | 1 | 2 |

TABLE 2
Sensitivity test result using standard strains (μg/ml)

| | | S. | S. | S. | E. |
|---|---|---|---|---|---|
| | Staphylococcus | aureus | aureus | epidermidis | faecalis |
| | aureus giorgio | 77 | K311 | R005 | EFS004 |
| 1-7 | <0.008 | 0.25 | 0.5 | 0.25 | 0.5 |
| 1-8 | 0.031 | 0.25 | 0.5 | 0.5 | 1 |
| I-9 | 0.031 | 0.25 | 1 | 0.5 | 1 |
| I-10 | 0.016 | 0.25 | 0.5 | 0.25 | 1 |
| I-11 | 0.016 | 0.25 | 0.5 | 0.5 | 2 |
| I-12 | 0.031 | 0.5 | 1 | 0.5 | 0.5 |
| I-13 | 0.016 | 0.25 | 0.5 | 0.25 | 1 |
| I-14 | <0.008 | 0.13 | 0.25 | 0.13 | 1 |
| I-15 | 0.031 | 0.5 | 0.5 | 0.5 | 1 |
| I-16 | <0.008 | 0.13 | 0.5 | 0.13 | 0.5 |
| I-17 | <0.008 | 0.25 | 0.5 | 0.25 | 0.5 |
| I-18 | 0.016 | 0.25 | 1 | 0.25 | 1 |
| Vancomycin | 1 | 1 | 2 | 1 | 2 |

While the invention has been described with respect to the above specific embodiments, it should be recognized that various modifications and changes can be made to the invention by those skilled in the art, which also fall within the scope of the invention as defined by the appended claims.

1. A cephalosporin compound represented by the following formula (I): 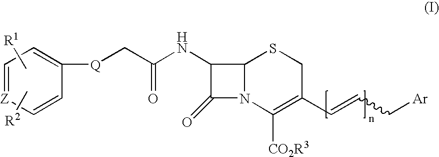
and pharmaceutically acceptable non-toxic salt, physiologically hydrolysable ester, hydrate, solvate or isomer thereof, in which
R¹ and R² independently of one another represent hydrogen, halogen, C₁₋₆ alkyl, C₁₋₆ alkylthio, aryl, arylthio, or C₅₋₆ heteroaryl containing one or two hetero atoms selected from the group consisting of nitrogen and oxygen;
R³ represents hydrogen or a carboxy-protecting group;
Q represents O, S, CH₂, NH or NR, wherein R represents hydrogen, C₁₋₆ alkyl or benzyl;
Z represents CH or N;
n denotes an integer of 0 or 1;
Ar represents a heteroaryl group represented by one of the following formulas: 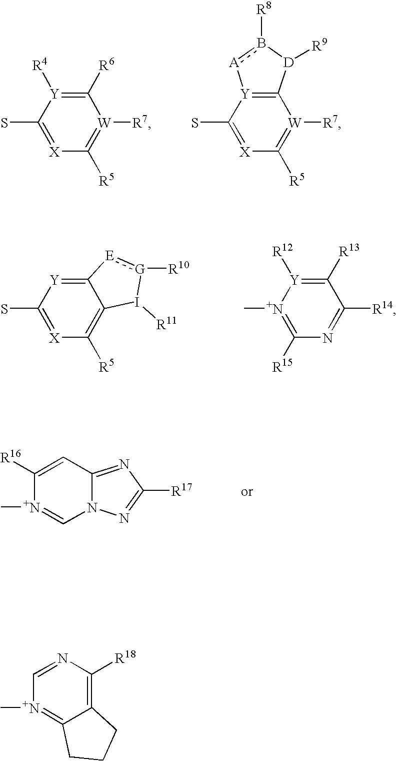
wherein X, Y, W, A, B, D, B, G and I independently of one another represent N or C (or CH), provided that the six-membered ring forms a pyrimidine structure;
R⁴ represents hydrogen or C₁₋₄ alkyl or amino substituted or unsubstituted with a substituent selected from the group consisting of C₁₋₆ alkyl and C₁₋₆ hydroxyalkyl;
R⁵ and R⁶ independently of one another represent hydrogen or hydroxy, or represent C₁₋₄ alkyl C₁₋₆ alkylthio or amino substituted or unsubstituted with a substituent selected from the group consisting of C₁₋₆ alkyl, C₁₋₆ hydroxyalkyl and C₁₋₆ aminoalkyl;
R⁷, R⁸, R⁹, R¹⁰ and R¹¹ independently of one another represent hydrogen, or represent C₁₋₆ alkyl or represent amino substituted or unsubstituted with a substituent selected from the group consisting of C₁₋₆ alkyl C₁₋₆ hydroxyalkyl and C₁₋₆ aminoalkyl;
R¹², R¹³, R¹⁴, R¹⁵, R¹⁶, R¹⁷ and R¹⁸ independently of one another represent hydrogen, C₁₋₆ alkyl or C₁₋₆ hydroxyalkyl, or represent amino substituted or unsubstituted with a substituent selected from the group consisting of C₁₋₆ alkyl, di-C₁₋₆ alkyl, C₁₋₆ hydroxyalkyl and C₁₋₆ aminoalkyl; [symbol]
denotes a single bond or a double bond; and
the propenyl group when n is 1 at C-3 position may be present in the form of cis or trans.

2. The compound of claim 1, wherein the compound is selected from the group consisting of the following:
(6R,7R)-3-{(E)-3-[(2-amino-6-hydroxy-4-pyrimidinyl)sulfanyl]-1-propenyl}-7-({2-[(2,5-dichlorophenyl)sulfanyl]acetyl }amino)-8-oxo-5-thia-1-azabicyclo[4.2.0]oct-2-ene-2-carboxylic acid;
4-amino-i -{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3 -yl]-2-propenyl}pyrimidin-1-ium;
(6R,7R)-3-{(E)-3-[(2,6-diamino-4-pyrimidinyl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid;
1,4-diamino-2-({(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}sulfanyl)pyrimidin-1-ium;
(6R,7R)-3-{(E)-3-[(2,6-diamino-4-pyrimidinyl)sulfanyl]-1-propenyl-7-{[2-(2,5-dichloroanilino)acetyl]amino}-8-oxo-5-thia-1-azabicyclo[4.2.0]oct-2-ene-2-carboxylic acid;
1,4-diamino-2-({[(6R,7R)-2-carboxy-7-({2-[(2,5-dichlorophenyl)sulfanyl]acetyl}amino)-8-oxo-5-thia-1-azabicyclo[4.2.0]oct-2-en-3 -yl]methyl}sulfanyl)pyrimidin-1-ium;
(6R,7R)-7-({2-[(2,6-dichloro-4-pyridinyl)sulfanyl]acetyl}amino)-8-oxo-3-[(E)-3-(1H-pyrazolo[3,4-d]pyrimidin-4-ylsulfanyl]-1-propenyl]-5-thia-1-azabicyclo[4.2.0]oct-2-ene-2-carboxylic acid;
(6R,7R)-3-{(E)-3-[(4,6-diamino-2-pyrimidinyl)sulfanyl]-1-propenyl}-7-({2-[(2,6-dichloro-4-pyridinyl)sulfanyl]acetyl}amino)-8-oxo-5-thia-1-azabicyclo[4.2.0]oct-2-ene-2-carboxylic acid;
(6R,7R)-3-{(E)-3-[(4-amino-1H-pyrazolo[3,4-d]pyrimidin-6-yl)sulfanyl]-1-propenyl}-7-({2-[(2,6-dichloro-4-pyridinyl)sulfanyl]acetyl}amino)-8-oxo-5-thia-1-azabicyclo[4.2.0]oct-2-ene-2-carboxylic acid;
(6R,7R)-3-{(E)-3-[(2-amino-6-hydroxy-4-pyrimidinyl)sulfanyl]-1-propenyl}-7-({2-[(2,6-dichloro-4-pyridinyl)sulfanyl]acetyl}amino)-8-oxo-5-thia-1-azabicyclo [4.2.0]oct-2-ene-2-carboxylic acid;
(6R,7R)-3-{(E)-3-[(2,6-diamino-4-pyrimidinyl)sulfanyl]-1-propenyl}-7-({2-[(2,6-dichloro-4-pyridinyl)sulfanyl]acetyl }amino)-8-oxo-5-thia-1-azabicyclo[4.2.0]oct-2-ene-2-carboxylic acid;
(6R,7R)-7-({2-[(2,6-dichloro-4-pyridinyl)sulfanyl]acetyl}amino)-3-((E)-3-{[2-(ethylsulfanyl)-6-methyl-4-pyrimidinyl]sulfanyl}-1-propenyl)-8-oxo-5-thia-1-azabicyclo[4.2.0]oct-2-ene-2-carboxylic acid;
7-amino-5-({(E)-3-[(6R,7R)-2-carboxy-7-({2-[(2,6-dichloro-4-pyridinyl)sulfanyl]acetyl}amino)-8-oxo-5-thia-1-azabicyclo[4.2.0]oct-2-en-3-yl]-2-propenyl}sulfanyl)-1H-[1,2,4]triazolo[1,5-c]pyrimidin-4-ium;
2,7-diamino-5-({(E)-3-[(6R,7R)-2-carboxy-7-({2-[(2,6-dichloro-4-pyridinyl)sulfanyl]acetyl}amino)-8-oxo-5-thia-1-azabicyclo[4.2.0]oct-2-en-3-yl]-2-propenyl}sulfanyl)-1-methyl-1H-[1 ,2,4]triazolo[1,5-c]pyrimidin-4-ium;
(6R,7R)-7-({2-[(2,6-dichloro-4-pyridinyl)sulfanyl]acetyl}amino)-3-[(E)-3-({4-hydroxy-6-[(2-hydroxyethyl)amino]-2-pyrimidinyl]sulfanyl}-1-propenyl]-8-oxo-5-thia-1-azabicyclo[4.2.0]oct-2-ene-2-carboxylic acid;
4,6-diamino-2-({(E)-3-[(6R,7R)-2-carboxy-7-({2-[(2,6-dichloro-4-pyridinyl)sulfanyl]acetyl}amino)-8-oxo-5-thia-1-azabicyclo[4.2.0]oct-2-en-3-yl]-2-propenyl}sulfanyl)-1-ethylpyrimidin-1-ium;
1,2-diamino-4-({(E)-3-[(6R,7R)-2-carboxy-7-({2-[(2,6-dichloro-4-pyridinyl)sulfanyl]acetyl}amino)-8-oxo-5-thia-1-azabicyclo[4.2.0]oct-2-en-3-yl]-2-propenyl}sulfanyl)-6,7-dihydro-5H-cyclopenta[d]pyrimidin-1-ium;
4,6-diamino-1-({(E)-3-[(6R,7R)-2-carboxy-7-({2-[(2,6-dichloro-4-pyridinyl)sulfanyl]acetyl}amino)-8-oxo-5-thia-1-azabicyclo[4.2.0]oct-2-en-3-yl]-2-propenyl}pyrimidin-1-ium;
(6R,7R)-7-amino-5-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}sulfanyl)-3H-[1,2,4]triazolo[1,5-c]pyrimidin-4-ium;
(6R,7R)-3-{(E)-3-[(4-amino-6,7-dihydro-5H-cyclopenta[d]pyrimidin-2-yl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid,
(6R,7R)-1,2-diamino-4-({(E)-3-[2-carboxy-7-({2-[(2,5-dichlorophenyl)sulfanyl]acetyl}amino-8-oxo-5-thia-1-azabicyclo[4.2.0]oct-2-en-3-yl-2-propenyl}sulfanyl)-6,7-dihydro-5H-cyclopenta[d]pyrimidin-1-ium;
(6R,7R)-2,6-diamino-4-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}sulfanyl) -1-methylpyrimidin-1-ium;
(6R,7R)-4,6-diamino-2-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}sulfanyl)-1-methyl-5-[(methylamino)methyl]pyrimidin-1-ium;
(6R,7R)-3-{(E)-3-[(4,6-diamino-2-pyrimidinyl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid;
(6R,7R)-3-{(E)-3-[(5,6-diamino-4-pyrimidinyl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid;
(6R,7R)-3-{(E)-3-[(4,6-diamino-5-methyl-2-pyrimidinyl)sulfanyl]-1-propenyl}-7-({2-[(2,5-dichlorophenyl)sulfanyl]acetyl}amino)-8-oxo-5-thia-1-azabicyclo[4.2.0]oct-2-ene-2-carboxylic acid;
(6R,7R)-7-({2-[(2,5-dichlorophenyl)sulfanyl]acetyl}amino)-8-oxo-3-[(E)-3-(1H-pyrazolo[3,4-d]pyrimidin-4-ylsulfanyl)-1-propenyl-5-thia-1-azabicyclo[4.2.0]oct-2-ene-2-carboxylic acid;
(6R,7R)-7-({2-[(2,5-dichlorophenyl)sulfanyl]acetyl}amino)-3-((E)-3-{[6-methyl-2-(methylsulfanyl)-4-pyrimidinyl]sulfanyl}-1-propenyl)-8-oxo-5-thia-1-azabicyclo [4.2.0]oct-2-ene-2-carboxylic acid;
(6R,7R)-4,6-diamino-2-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}sulfanyl) -1-ethylpyrimidin-1-ium;
(6R,7R)-3-((E)-3-{[4-amino-6-(methylamino)-2-pyrimidinyl]sulfanyl}-1-propenyl)-7-({2-[(2,5-dichlorophenyl)sulfanyl]acetyl }amino)-8-oxo-5-thia-1-azabicyclo [4.2.0]oct-2-ene-2-carboxylic acid;
(6R,7R)-4,6-diamino-1-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-5-methylpyrimidin-1-ium;
(6R,7R)-2,7-diamino-6-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-[1,2,4]triazolo[1,5-c]pyrimidin-6-ium;
(6R,7R)-4-amino-1-({(E)-3-[2-carboxy-7-({2-[(2,5-dichlorophenyl)sulfanyl]acetyl}amino)-8-oxo-5-thia-1-azabicyclo[4.2.0]oct-2-en-3 -yl]-2-propenyl}-2-methyl pyrimidin-1-ium;
(6R,7R)-4-amino-1-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-6,7-dihydro-5H-cyclopenta[d]pyrimidin-1-ium;
(6R,7R)-4,5,6-triamino-1-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}pyrimidin-1-ium;
(6R,7R)-4,6-diamino-1-({(E)-3-[2-carboxy-7-({2-[(2,5-dichlorophenyl)sulfanyl]acetyl}amino)-8-oxo-5-thia-1-azabicyclo[4.2.0]oct-2-en-3-yl]-2-propenyl}pyrimidin-1-ium;
4-amino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-6-(dimethylamino)-2-methylpyrimidin-1-ium;
4-amino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl }-2-methyl-6-(m ethylamino)pyrimidin-1-ium;
4-amino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-2-methyl pyrimidin-1-ium;
4,6-diamino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-5-methylpyrimidin-1-ium; and
4-amino-1-{(E)-3-[(6R,7R)-2-carboxy-7-({2-[(2,5-dichlorophenyl)sulfanyl]acetyl}amino)-8-oxo-5-thia-1-azabicyclo[4.2.0]oct-2-en-3-yl]-2-propenyl}-2-methyl-6-(m ethylamino)pyrimidin-1-ium.

3. A process for preparing the compound of formula (I) according to claim 1, which comprises reacting a compound of formula (V): 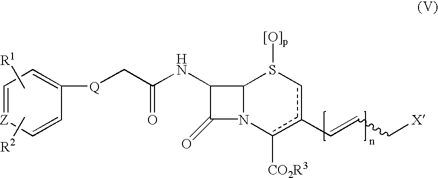
wherein R¹, R², R³, Z, Q and n are as defined in claim 1, X′ represents halogen atom, and p is 0 or 1, with a compound of formula (VI):
H—Ar (VI)
wherein Ar is as defined in claim 1, or reducing S→oxide of a compound of formula (VII): 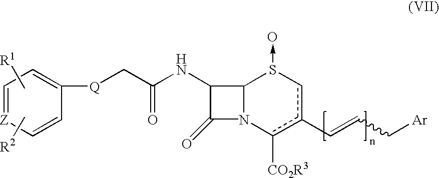
wherein R¹, r², R³, Z, Q, n and Ar are as defined in claim 1.

4. The process of claim 3, which further comprises removing acid-protecting group.

5. An antibacterial composition containing the compound of formula (I) or its pharmaceutically acceptable salt according to claim 1 as an active ingredient, together with a pharmaceutically acceptable carrier.
